(19) United States
(12) Patent Application Publication
(10) Pub. No.: US 2024/0166691 A1
(43) Pub. Date: May 23, 2024

(54) PRODRUGS AND COMPOSITIONS FOR OPHTHALMOLOGY APPLICATIONS

(71) Applicant: Alcon Inc., Fribourg (CH)

(72) Inventors: Howard Allen Ketelson, Dallas, TX (US); Rekha Rangarajan, Fort Worth, TX (US)

(21) Appl. No.: 18/503,390

(22) Filed: Nov. 7, 2023

Related U.S. Application Data
(60) Provisional application No. 63/382,793, filed on Nov. 8, 2022.

(51) Int. Cl. C07K 7/64

(52) U.S. Cl. CPC C07K 7/645

(57) ABSTRACT

The present disclosure is directed to prodrugs, compositions including prodrugs, and methods for administering prodrugs and compositions thereof to a patient, including prodrugs represented by Formula (I):

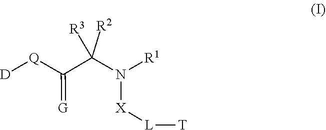

wherein: D is a drug moiety; Q is an oxygen atom, a sulfur atom, or a nitrogen atom; X is a chemical bond or

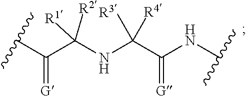

each of G, G′, and G″ is independently an oxygen atom or a sulfur atom; L is a polyether; T is a hydrogen atom, a hydroxyl group, a thiol group, a boronic acid group, or an amine group; R¹ is selected from the group consisting of a substituted aryl group, unsubstituted aryl group, and a substituted carbonyl group; and each of R², R³, R^{1′}, R^{2′}, R^{3′}, and R^{4′} is independently a hydrogen atom, a substituted C¹-C²⁰ alkyl group, or an unsubstituted C¹ -C²⁰ alkyl group. In at least one embodiment, a composition comprises a compound and a carrier material.

### FIELD

This application claims the benefit under 35 USC § 119 (e) of U.S. provisional application number 63/382,793 filed 8 Nov. 2022, herein incorporated by reference in its entirety.

The present disclosure is directed to prodrugs, compositions including prodrugs, and methods for administering prodrugs and compositions thereof to the eye of a patient.

### BACKGROUND

Ocular diseases and disorders, in particular those affecting the ocular surface, are widespread. For example, dry eye disease (DED), also known as Keratoconjunctivitis Sicca (KCS), is one of the most common ophthalmic disorders. Patients who visit ophthalmic clinics frequently report symptoms of dry eye, making it a growing public health problem and one of the most common conditions seen by eye care practitioners. Its prevalence increases significantly with age.

DED is a multifactorial disorder of the tear film and ocular surface that may result in eye discomfort symptoms such as dryness, burning sensation, itching, redness, stinging, blurred vision, grittiness, pain, visual disturbances, tear film instability, ocular fatigue, and often ocular surface damage. DED can also make it difficult or impossible for a patient to wear contact lenses, read, work on a computer, or drive at night.

Inflammation of both the lacrimal gland and ocular surface has been shown to play a role in dry eye. Factors that adversely affect tear film stability and osmolarity can induce ocular surface damage and initiate an inflammatory cascade that generates innate and adaptive immune responses. These immunoinflammatory responses lead to further ocular surface damage and the development of a self-perpetuating inflammatory cycle. For instance, inflammation of the ocular surface results in a reduction of tear production, which further deteriorates the conditions and potentially leads in turn to inflammation of ocular surface and epithelial cell damage.

DED can be categorized as acute, episodic, or chronic. In some cases, it can be categorized as chronic with acute flares. Chronic DED can require year-round attention. Several pharmacological therapies for DED have been explored and include a stepped approach starting with over the counter lubricants and artificial tear replacements (delivered as drops), progressing to topical anti-inflammatory therapy and lacrimal occlusion using punctal plugs to block tear drainage.

Artificial tears increase the tear volume, but the tear volume may return to its original state due to tear drainage and fluid loss by, e.g., evaporation or absorption through ocular epithelia, and thus require frequent administration. While the residence time could be increased by addition of viscosity enhancers, a high viscosity tear replacement may cause blurred vision. Residence time of eye drop formulations can also be important for treatment of glaucoma, presbyopia, myopia, and so called “back of the eye” diseases where treatment is difficult unless sufficient drug retention is achieved at the back of the eye. Although punctal plugs have been shown to be effective in patients with DED, plugs can be lost (show poor retention) and may rarely migrate into the nasolacrimal duct, resulting in inflammation or other critical conditions. In some cases, the punctum can be surgically closed with high temperature cautery in an effort to treat DED. Additional approved therapies for DED patients in US are Restasis® (cyclosporine) which increases tear production, Cequa® (cyclosporine) which increases tear production, and Xiidra® (lifitegrast) for signs and symptoms of DED. Eysuvis™ (loteprednol) has also been approved for acute treatment of DED.

In addition, cyclosporine A is a cyclic polypeptide calcineurin inhibitor immunosuppressant/immunomodulatory agent found in soil fungi, and its immunomodulatory activity is used in the treatment of immune-based disorders, such as transplant rejection, psoriasis, ulcerative colitis, and rheumatoid arthritis. Calcineurin is an enzyme that activates T-cells, which play a key role in cell-mediated immunity. Because calcineurin inhibitors suppress the immune system they are known as immunosuppressants.

Topical administration of cyclosporine A has been shown to increase tear fluid secretion, possibly by promoting the local release of parasympathetic nervous system-associated neurotransmitters. This is thought to occur through suppression of inflammation by cyclosporine on the ocular surface. In the management of KCS in dogs, the mechanism by which cyclosporine causes an increase in lacrimation is poorly understood, but clinical improvement is considered to be not necessarily dependent on an increase in tear production. In humans, the beneficial effects of cyclosporine A treatment in DED are better established and findings of several clinical trials indicate that long-term treatment with topical cyclosporine can yield positive results with regard to, for example, corneal surface staining, Schirmer test, blurred vision, frequency of artificial tear application, and also with respect to cellular and molecular markers of disease severity.

Cyclosporine for ophthalmic use was first approved in 1995 for the treatment of KCS in dogs. In 2003, it was approved for ophthalmic use in humans as Restasis® (cyclosporine ophthalmic emulsion 0.5 mg/mL, Allergan) and is indicated for increased tear production in patients whose tear production is presumed to be suppressed due to ocular inflammation associated with KCS. Topical cyclosporine eye drops were shown to decrease inflammatory mediators and increase tear production. Commercial and marketed topical cyclosporine eye drops are sold around the world for the treatment of DED/KCS, Vernal Keratoconjunctivitis (VKC), and ocular inflammation. Cyclosporine ophthalmic solution is currently on the market for topical use for multiple products in multiple jurisdictions.

However, there are limitations with the application of topical drops, which affect patient management. Such limitations can include difficulty with handling the bottle, limited instillation accuracy, potential washout of drops, and limited bioavailability of topical eye drops. Limitations with currently available eye drop formulations of cyclosporine include tolerability issues such as burning and stinging. The high frequency of administration (e.g. several times per day) can highly affect daily life of patients. In humans, the bioavailability from topical eye drops reaching the ocular tissues is less than 5%. Bioavailability of topically applied cyclosporine is a result of complex processes such as (1) precorneal clearance factors (e.g., due to blinking and lacrimation), (2) tear film drug concentration time curve (e.g., amount of cyclosporine in the tears), (3) tissue permeability, and (4) post tissue clearance. Other limitations include a delayed onset of action (many weeks to months), as well as the high drug dose in the drops, which may be the cause for adverse reactions, such as ocular burning associated with topical cyclosporine eye drops.

As mentioned above, in order to improve the retention time of drugs in general at an ocular surface, mucoadhesive polymers can be used, and these polymers rely on the formation of non-covalent bonds such as hydrogen bonds, ionic bonds, or van der waals forces to interact with the ocular surface. In addition to causing blurry vision, such polymers providing hydrogen bonds, van der waals forces, etc. are promoting bonds/forces that are transient (easily reversible) and very ineffective unless multiple dosing posology is used. Since the cornea is the major route of entry for eye drop drugs there are several challenges. The superficial surface cell layer has tight junctions and is typically rate limiting for drug adsorption. Further, the tear turnover dilutes drugs on contact and limits the residence time for a drug to absorb. These two mechanisms have considerable difference in kinetics. Mechanisms of absorption include passive diffusion through and around cells as well as active receptor and vesicle mediated transport. Hydrophobic small molecules adsorb transcellularly, and permeate best when they are low molecular weight, lipophilic, and not charged. However, hydrophilic small molecules and macromolecules in general absorb paracellularly and much more slowly.

Alternatively, drug delivery from punctal plugs are beneficial over topical drops in that they allow for a sustained release of the drug over time by forming a depot from which the drug is slowly being released. Administration consists of a one-time administration of the plug, which addresses the above-mentioned limitations related to long-term administration of topical eye-drops. However, the intracanalicular administration route has certain anatomically implied restrictions (it needs to be small enough to enter the lacrimal punctum) and it is difficult to develop an ophthalmic intracanalicular plug that is easy to administer and to remove once the drug depot is depleted if necessary, fits well, i.e. provides appropriate retention so that it is not unintentionally lost, but at the same time does not cause any discomfort or unintended administration site reactions such as inflammation. In addition, drug release needs to be appropriate and consistent over a sustained period of time. In view of the small size of the plug, it is challenging to formulate to include an adequate drug load and sustained-release properties.

There is a need in the art for topically administered ophthalmic drug delivery at reduced concentrations, as compared to a conventional formulation, and improvement of adverse reactions associated with conventional topically administered ophthalmic drug delivery. There is further a need for topically administered ophthalmic drug delivery systems having sustained viability with reduced frequency of application of the drug topically to a patient.

### SUMMARY

The present disclosure is directed to prodrugs, compositions including prodrugs, and methods for administering prodrugs and compositions thereof to a patient.

In at least one embodiment, a compound is represented by Formula (I):

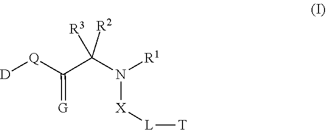

wherein:
D is a drug moiety;
Q is an oxygen atom, a sulfur atom, or a nitrogen atom;
G is an oxygen atom or a sulfur atom;
X is a chemical bond or

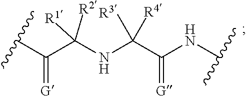

L is a polyether, polythioether, acetal, ester, hydrazone, imine, orthoester, oxime, phosphoramidate, vinyl ether, or β-thiopropionate;
T is a hydrogen atom, a hydroxyl group, a thiol group, a boronic acid group, or an amine group;
R¹ is selected from the group consisting of a substituted aryl group, unsubstituted aryl group, and a substituted carbonyl group; and
each of R² and R³ is independently a hydrogen atom, a substituted C¹-C²⁰ alkyl group, or an unsubstituted C¹-C^{°} alkyl group.

In at least one embodiment, a composition comprises a compound and a carrier material.

### BRIEF DESCRIPTION OF THE DRAWINGS

FIG. 1 is a graph illustrating photometrical analysis the solubility of pH-sensitive CsA-PEG-2K derivative (I), according to an embodiment.

FIG. 2 is a graph illustrating half-life time of pH-sensitive CsA-PEG-2K, according to an embodiment.

FIG. 3 is a graph illustrating stability of pH-sensitive CsA-PEG-2K derivative (I) at different temperatures, according to an embodiment.

### DETAILED DESCRIPTION

### Definitions

As used herein, a “prodrug” is a compound that, after being administered to a subject, is metabolized into a pharmacologically active drug, for example, by releasing a drug compound. The prodrug itself need not be pharmacologically active, and preferably is not pharmacologically active.

As used herein, the terms “administer(s)” “administered”, “administering” refers to the placement of a composition into a subject by a method or route which results in at least partial localization of the composition at a desired site such that desired effect is produced. A compound (e.g., drug or prodrug) or composition of the present disclosure can be administered by any appropriate route including, but not limited to, topical administration (e.g., ophthalmic drops).

As used herein, the terms “carrier” and “pharmaceutically acceptable carrier” may be used interchangeably, and mean any liquid, suspension, gel, salve, solvent, liquid, diluent, fluid ointment base, nanoparticle, liposome, or micelle, which is suitable for use in contact with a subject without causing adverse physiological responses, and which does not interact with the other components of the composition in a way that would reduce efficacy of a drug or prodrug of the composition. A number of carrier ingredients can be used for making topical compositions, such as gelatin, polymers, fats and oils, lecithin, collagens, alcohols, water, etc. The term “pharmaceutically acceptable” means those compounds, materials, compositions, and dosage forms which are, within the scope of sound medical judgment, suitable for use in contact with the tissues of a subject without excessive toxicity, irritation, or other problem or complication, commensurate with a reasonable benefit/risk ratio.

As used herein, the term “isomer(s)” means all stereoisomers of the compounds and/or molecules referred to herein (e.g., Cyclosporine A), including enantiomers, diastereomers, as well as all conformers, rotamers, and tautomers, unless otherwise indicated. The compounds and/or molecules disclosed herein include all enantiomers in either substantially pure levorotatory or dextrorotatory form, or in a racemic mixture, or in any ratio of enantiomers. Where embodiments disclose a (D)-enantiomer, that embodiment also includes the (L)-enantiomer; where embodiments disclose a (L)-enantiomer, that embodiment also includes the (D)-enantiomer. Where embodiments disclose a (+)-enantiomer, that embodiment also includes the (−)-enantiomer; where embodiments disclose a (−)-enantiomer, that embodiment also includes the (+)-enantiomer. Where embodiments disclose an (S)-enantiomer, that embodiment also includes the (R)-enantiomer; where embodiments disclose an (R)-enantiomer, that embodiment also includes the (S)-enantiomer. Embodiments are intended to include any diastereomers of the compounds and/or molecules referred to herein in diastereomerically pure form and in the form of mixtures in all ratios. Unless stereochemistry is explicitly indicated in a chemical structure or chemical name, the chemical structure or chemical name is intended to embrace all possible stereoisomers, conformers, rotamers, and tautomers of compounds and/or molecules depicted.

As used herein, the terms “treat”, “treating”, or “treatment(s)” means the application or administration of a composition of the present disclosure, or identified by a method of the present disclosure, to a subject, or application or administration of the therapeutic agent to an isolated tissue or cell line from a subject, who has a disease, a symptom of disease, or a predisposition toward a disease, with the purpose to cure, heal, alleviate, relieve, alter, remedy, ameliorate, improve, or affect the disease, the symptoms of disease, or the predisposition toward disease. As used herein, the term “subject” refers to an animal, such as a mammal, including for example a human or domesticated animal (e.g., a dog or cat), which is to be the recipient of a particular treatment.

As used herein, terms “effective amount”, “therapeutically effective amount”, or “pharmaceutically effective amount” may be used interchangeably and mean the amount of a compound that, when administered to a subject for treating a state, disorder or condition, is sufficient to effect such treatment. The “effective amount” will vary depending on the compound, the disease and its severity, and the age, weight, physical condition and responsiveness of the mammal to be treated.

As used herein, the terms “ocular” or “ocular region” means the eye, surrounding tissues of the eye, and to bodily fluids in the region of the eye. Specifically, the term includes the cornea or, the sclera or, the uvea, the conjunctiva (e.g., bulbar conjunctiva, palpebral conjunctiva, and tarsal conjunctiva), anterior chamber, lacrimal sac, lacrimal canals, lacrimal ducts, medial canthus, nasolacrimal duct, and the eyelids (e.g., upper eyelid and lower eyelid). Additionally, the term includes the inner surface of the eye (conjunctiva overlying the sclera), and the inner surface of the eyelids (e.g., the palpepral conjunctiva).

As used herein, “ocular surface” includes the conjunctiva and/or the cornea, together with elements such as the lacrimal apparatus, including the lacrimal punctum, as well as the lacrimal canaliculus and associated eyelid structures.

As used herein, the term “conjunctiva” means the mucous membrane lining the inner surfaces of the eyelids and anterior part of the sclera.

As used herein, the term “cornea” means the clear central frontal tissue of the eye. The degree of corneal curvature varies from subject to subject.

As used herein, the term “eye(s)” means the light sensing organs of a subject and can refer to the sense organ providing vision to a subject.

As used herein, the term “eyelid” means a movable fold of thin skin over the eye, which may further comprise eyelashes and ciliary and meibomian glands along its margin. The eyelid includes loose connective tissue containing a thin plate of fibrous tissue lined with mucous membrane (conjunctiva).

As used herein, the term “canthus” means either corner of the eye where the upper and lower eyelids meet.

As used herein, the term “mucus” means the viscous, slippery secretions of mucous membranes and glands, containing mucin, white blood cells, water, inorganic salts, and exfoliated cells.

As used herein, the term “lacrimal apparatus” refers to one or more of a lacrimal gland, lacrimal duct, lacrimal sac, or lacrimal canal, or any organ associated with the production or drainage of tears.

As used herein, the term “sclera” means the collagenous outer-wall of the eyeball comprising mostly collagen and some elastic tissue, which is covered by conjunctiva. In humans, the sclera is sometimes referred to as the white of the eye.

As used herein, the term “tear(s)” or “tear fluid” means the liquid produced by lacrimation, for cleaning and lubricating the eyes. Tears include liquid secreted by the lacrimal glands, which lubricates the eyes and thus forms the tear film. Tears are made up of water, electrolytes, proteins, lipids, and mucins.

The term “sustained release” is meant to characterize products which are formulated to make a drug compound available over an extended period of time, thereby allowing a reduction in dosing amount and/or frequency compared to an immediate release dosage form, such as a solution of the drug compound that is topically applied onto the eye (e.g., cyclosporine-containing eye drops). Other terms that may be used herein interchangeably with “sustained release” are “extended release” or “controlled release”.

### Prodrugs, Compositions, and Methods Thereof

The present disclosure provides prodrugs, compositions including prodrugs, and methods for administering prodrugs and compositions thereof to the eye of a patient. Prodrugs, compositions, and methods of the present disclosure can provide treatments of ocular disorders of the eye, such as ocular surface diseases, as well as those associated with inflammation of internal cells and cell layers of the eye, and including diseases of the retina and other back of the eye diseases. Methods include methods for treating ocular disorders, particularly dry eye disorders, by topically administering to an ocular region of a subject a composition comprising a pharmaceutically effective amount of at least one prodrug and a pharmaceutically acceptable carrier. Methods can include reducing or preventing one or more symptoms or causes of dry eye.

Prodrugs, compositions, and methods of the present disclosure provide topically administered ophthalmic drug delivery at reduced concentrations, as compared to conventional formulations and improvement of adverse reactions associated with conventional topically administered ophthalmic drug delivery. Prodrugs, compositions, and methods of the present disclosure further provide topically administered ophthalmic drug delivery systems having sustained viability (e.g., release of a drug compound from the prodrug) with reduced frequency of application of the drug compound topically to a patient.

In addition, prodrugs of the present disclosure provide reduced “lead in” time even at reduced concentration of drug content.

### Prodrugs

Prodrugs of the present disclosure can enhance ocular retention, and hence enhance exposure time of the a drug to the ocular surface. A prodrug of the present disclosure can be configured to undergo an intramolecular cyclization reaction between a nitrogen atom and a carbonyl group to form a piperazine-dione and release a drug compound that is bound to the prodrug (before the intramolecular cyclization). In some embodiments, a piperazine-dione is a piperazine-2,5-dione.

In some embodiments, prodrugs of the present disclosure have (1) a moiety which upon instillation on the ocular surface forms a covalent bond with molecules of the ocular surface, (2) a drug moiety to provide a desired pharmacological activity upon release from the prodrug, and (3) a labile spacer that links the moiety binding the ocular surface and the drug moiety. As an example, the moiety that forms a covalent bond with molecules of the ocular surface can be an active thiol moiety or boronic acid moiety or maleimide moiety. Thiol moieties can form disulfide bonds with cysteine-containing subdomains of mucus glycoproteins commonly found as soluble and bound ocular mucins. Boronic acid bind to the cis-diol groups in the mucin layer via boronate ester linkages. Thiols and Malemide bind to the sulfhydryl group on mucins to form disulfide linkages. The labile spacer is cleavable and is sensitive to tear film pH and enzymatic hydrolysis. In addition, because many drugs (such as cyclosporine A) are lipophilic, a labile spacer can include a group (such as polyethylene glycol) to provide improved hydrophilic properties for improved solubilization and bioavailability of the drug. The labile spacer can further include a pH dependent moiety that is configured to cleave an adjacent drug moiety under pH conditions of tear fluid and release the drug moiety (as a drug molecule) for treatment of the ocular surface. The pH dependent moiety cleaves the adjacent drug moiety via intramolecular cyclization to form a piperazine-dione.

Prodrugs of the present disclosure provide tethering of a drug moiety on the ocular surface of a patient to avoid dilution/removal from the ocular surface by tear fluid. Tethering the drug moiety on the ocular surface provides reduced concentration of active drug needed to achieve a desirable drug concentration (such as a Cmax) at the ocular surface. The reduced concentration of active drug provides improved tolerance by a patient (reduced adverse effects) and improved outcomes for dry eye therapy.

In alternative embodiments, prodrugs of the present disclosure do not have a moiety that forms a covalent bond with molecules of the ocular surface. Nonetheless, a labile spacer which is present can render the drug moiety inactive until the labile spacer is reacted and removed by the pH conditions of tear fluid. Such embodiments can provide a delayed release of the drug moiety for sustained administration of the drug moiety over extended periods of time, as compared to administering the drug moiety as a single dose.

In some embodiments, a prodrug is represented by Formula (I):

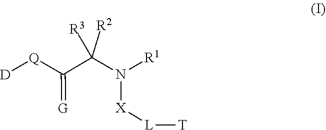

wherein:
D is a drug moiety;
Q is an oxygen atom, a sulfur atom, or a nitrogen atom;
X is a chemical bond or

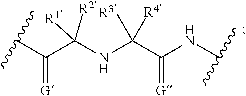

each of G, G′, and G″ is independently an oxygen atom or a sulfur atom;
L is a polyether, polythioether, acetal, ester, hydrazone, imine, orthoester, oxime, phosphoramidate, vinyl ether, or P-thiopropionate;
T is a hydrogen atom, a hydroxyl group, a thiol group, a boronic acid group, or an amine group;
R¹ is a substituted or unsubstituted aryl group (including aralkyl or alkaryl) or a substituted carbonyl group; and
each of R², R³, R^{2′}, R^{3′}, and R^{4′} is independently a hydrogen atom or substituted or unsubstituted C¹-C²⁰ alkyl.

It is understood that Q of Formula (I) is an atom provided by a drug molecule of D during the formation of the prodrug of Formula (I). In other words, the drug molecule that forms drug moiety D also provides atom Q during the formation of the prodrug of Formula (I). For example, for producing a prodrug where D is cyclosporine A, a drug molecule that is cyclosporine A provides the cyclosporine main structure as drug moiety D as well as an oxygen atom as atom Q of the prodrug of Formula (I). In another example, for producing a prodrug where D is lifitegrast, a drug molecule that is lifitegrast provides the lifitegrast main structure as drug moiety D as well as an oxygen atom as atom Q of the prodrug of Formula (I).

In some embodiments, R¹ is an aryl group selected from substituted or unsubstituted phenyl or substituted or unsubstituted benzyl. In at least one embodiment, R¹ is unsubstituted benzyl. In some embodiments, R¹ is a methyl acyl group substituted at the methylene carbon of the methyl acyl group with an amine group that, once formed, is a secondary amine group. The methylene carbon of the methyl acyl group can be additionally substituted independently with one or two alkyl groups, such as methyl, ethyl, propyl, butyl, pentyl, hexyl, or isomers thereof.

In some embodiments, each of R², R³, R^{1′}, R^{2′}, R^{3′}, and R^{4′}is independently a hydrogen atom, methyl, ethyl, propyl, butyl, pentyl, hexyl, heptyl, octyl, nonyl, or decyl.

In some embodiments, R¹ is selected from the group consisting of:

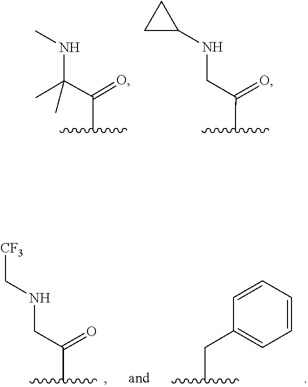

In some embodiments, X is

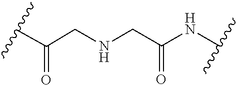 [SMILES: *NC(=O)CNCC(*)=O]

### and R¹ is

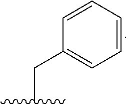 [SMILES: *Cc1ccccc1]

In some embodiments, X is a chemical bond and R¹ is selected from the group consisting of

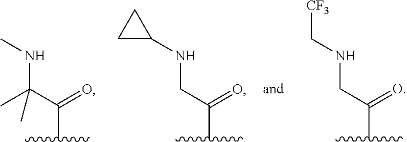

In some embodiments, L is a polyether that is a polyethylene glycol or a polypropylene glycol. In at least one embodiment, L is a polyethylene glycol. A polyethylene glycol can have any suitable amount of ethylene oxide units, such as about 2 to about 4,000 ethylene oxide units, such as about 500 to about 3,000 ethylene oxide units, such as about 1,500 to about 2,500 ethylene oxide units, such as about 2,000 ethylene oxide units. A polypropylene glycol can have any suitable amount of propylene oxide units, such as about 2 to about 4,000 propylene oxide units, such as about 500 to about 3,000 propylene oxide units, such as about 1,500 to about 2,500 propylene oxide units, such as about 2,000 propylene oxide units. It should be noted that for those skilled in the art that the L chain length may vary to optimize the physico-chemical properties such as solubility and stability. In some embodiments, a prodrug is represented by Formula (II):

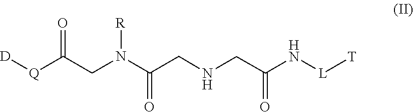

wherein:
D is a drug moiety;
Q is an oxygen atom, a sulfur atom, or a nitrogen atom;
L is a polyether, polythioether, acetal, ester, hydrazone, imine, orthoester, oxime, phosphoramidate, vinyl ether, or β-thiopropionate;
T is a hydrogen atom, a hydroxyl group, a thiol group, a boronic acid group, or an amine group; and
R is an aryl group.

It is understood that Q of Formula (II) is an atom provided by a drug molecule of D during the formation of the prodrug of Formula (II). In other words, the drug molecule that forms drug moiety D also provides atom Q during the formation of the prodrug of Formula (II). For example, for producing a prodrug where D is cyclosporine A, a drug molecule that is cyclosporine A provides the cyclosporine main structure as drug moiety D as well as an oxygen atom as atom Q of the prodrug of Formula (II). In another example, for producing a prodrug where D is lifitegrast, a drug molecule that is lifitegrast provides the lifitegrast main structure as drug moiety D as well as an oxygen atom as atom Q of the prodrug of Formula (II).

In some embodiments of Formula (II), R is an aryl group selected from substituted or unsubstituted phenyl or substituted or unsubstituted benzyl. In at least one embodiment, R is unsubstituted benzyl. In some embodiments of Formula (II), R is

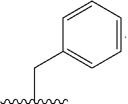 [SMILES: *Cc1ccccc1]

In some embodiments of Formula (II), L is a polyether that is a polyethylene glycol or a polypropylene glycol. In at least one embodiment, L is a polyethylene glycol. A polyethylene glycol can have any suitable amount of ethylene oxide units, such as about 2 to about 4,000 ethylene oxide units, such as about 500 to about 3,000 ethylene oxide units, such as about 1,500 to about 2,500 ethylene oxide units, such as about 2,000 ethylene oxide units. A polypropylene glycol can have any suitable amount of propylene oxide units, such as about 2 to about 4,000 propylene oxide units, such as about 500 to about 3,000 propylene oxide units, such as about 1,500 to about 2,500 propylene oxide units, such as about 2,000 propylene oxide units.

In some embodiments of Formula (II), T is a hydrogen atom, a boronic acid group, or a thiol group. In at least one embodiment, T is —SH.

In some embodiments, a prodrug is represented by Formula (III):

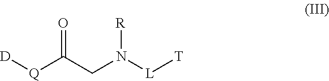

wherein:
D is a drug moiety;
Q is an oxygen atom, a sulfur atom, or a nitrogen atom;
L is a polyether, polythioether, acetal, ester, hydrazone, imine, orthoester, oxime, phosphoramidate, vinyl ether, or β-thiopropionate;
T is a hydrogen atom, a hydroxyl group, a thiol group, a boronic acid group, or an amine group; and
R is a substituted carbonyl group.

It is understood that Q of Formula (III) is an atom provided by a drug molecule of D during the formation of the prodrug of Formula (III). In other words, the drug molecule that forms drug moiety D also provides atom Q during the formation of the prodrug of Formula (III). For example, for producing a prodrug where D is cyclosporine A, a drug molecule that is cyclosporine A provides the cyclosporine main structure as drug moiety D as well as an oxygen atom as atom Q of the prodrug of Formula (III). In another example, for producing a prodrug where D is lifitegrast, a drug molecule that is lifitegrast provides the lifitegrast main structure as drug moiety D as well as an oxygen atom as atom Q of the prodrug of Formula (III).

In some embodiments of Formula (III), R is a methyl acyl group substituted at the methylene carbon of the methyl acyl group with an amine group that, once formed, is a secondary amine group. The methylene carbon of the methyl acyl group can be additionally substituted independently with one or two alkyl groups, such as methyl, ethyl, propyl, butyl, pentyl, hexyl, or isomers thereof.

In some embodiments of Formula (III), R is represented by formula (IIIa):

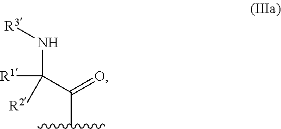

where R^{1′} and R^{2′} are independently hydrogen or substituted or unsubstituted C₁-C₁₀ alkyl, and R^{3′} is substituted or unsubstituted aryl or substituted or unsubstituted alkyl. In some embodiments of Formula (IIIa), R^{1′} and R^{2′} are each hydrogen. Alternatively, R^{1′} and R^{2′} are independently C₁-C₅ alkyl, such as methyl or ethyl. In some embodiments, one of R^{1′} or R^{2′} is hydrogen and the other of R^{1′} or R^{2′} is C₁-C₅, such as methyl or ethyl. In some embodiments of Formula (IIIa), R^{3′} is unsubstituted C₁-C₁₀ alkyl, such as methyl, ethyl, n-propyl, isopropyl, cyclopropyl, n-butyl, isobutyl, secbutyl, n-pentyl, isopentyl, secpentyl, or neopentyl. In some embodiments of Formula (IIIa), R^{3′} is C₁-C₁₀ alkyl substituted with one or more electron donating groups or electron withdrawing groups. For example, an electron donating group can be a C₁-C₁₀ alkoxy group. An electron withdrawing group can be a halogen or a nitro group. In some embodiments, of Formula (IIIa), R^{3′} is a C₁-C₁₀ alkyl substituted with one, two, three, four, or five fluorine atoms.

In some embodiments of Formula (III), R is selected from the group consisting of:

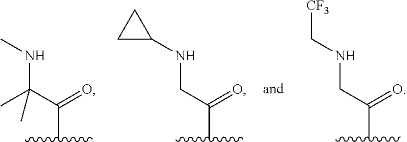

In some embodiments of Formula (III), L is a polyether that is a polyethylene glycol or a polypropylene glycol. In at least one embodiment, L is a polyethylene glycol. A polyethylene glycol can have any suitable amount of ethylene oxide units, such as about 2 to about 4,000 ethylene oxide units, such as about 500 to about 3,000 ethylene oxide units, such as about 1,500 to about 2,500 ethylene oxide units, such as about 2,000 ethylene oxide units. A polypropylene glycol can have any suitable amount of propylene oxide units, such as about 2 to about 4,000 propylene oxide units, such as about 500 to about 3,000 propylene oxide units, such as about 1,500 to about 2,500 propylene oxide units, such as about 2,000 propylene oxide units.

In some embodiments of Formula (III), T is a hydrogen atom, a boronic acid group, or a thiol group. In at least one embodiment, T is —SH.

The chemical units and molecular weight of an L group of Formulas (I), (II), or (III) can be chosen depending on the identity of drug moiety D. For example, a lipophilic drug moiety may have an L group that is hydrophilic and has a relatively high molecular weight. In other embodiments, a hydrophilic drug moiety may have an L group that has a relatively low molecular weight.

In some embodiments of Formulas (I), (II), or (III), the prodrug has a t_{1/2} (at pH=7.4-7.9) of about 90 minutes or less, such as about 60 minutes or less, such as about 30 minutes or less. t_{1/2} is defined as the amount of time that 1/2 of the amount of prodrug has reacted to release drug moiety (D) as an independent molecule from the remainder of the prodrug molecule. The t_{1/2} can be tuned depending on the electron donating or electron withdrawing content of the prodrug. For example, a prodrug of Formulas (I), (II), or (III) having electron withdrawing substitutions can have a longer t_{1/2}, as compared to a prodrug of Formulas (I), (II), or (III) having electron donating substitutions, because the nucleophilic nature of a nitrogen atom of the prodrug has been reduced due to the presence of electron withdrawing groups. Likewise, a prodrug of Formulas (I), (II), or (III) having electron donating substitutions can have a shorter t_{1/2} as compared to a prodrug of Formulas (I), (II), or (III) having electron withdrawing substitutions, because the nucleophilic nature of a nitrogen atom of the prodrug has been increased due to the presence of electron donating groups.

### Drug Moieties

Drug moieties of the present dislcosure (such as D of Formula (I)) can be any suitable drug moiety for administration to an ocular surface. To form a drug moiety, a drug molecule is reacted with a labile spacer of the present disclosure and the drug molecule becomes a drug moiety bonded to the labile spacer which collectively forms a prodrug of the present disclosure. A drug molecule can have a functional group, such as a hydroxyl group, a thiol group, or an amine group, that is capable of reacting with a labile spacer. In some embodiments, a drug molecule has one hydroxyl group or one thiol group. In some cases there may be multiple groups which can be selectively protected if needed.

In some embodiments, a drug molecule is cyclosporine A (CsA). CsA has a cyclic undecapeptide structure and has one functional group (hydroxyl group) on the 2-N-methyl-(R)-((E)-2-butenyl)-4-methyl-L-threonine residue. The hydroxyl group can be utilized to react with a labile spacer to form a drug moiety of a prodrug of the present disclosure.

The molecular formula of cyclosporine A is C₆₂H₁₁₁N₁₁O₁₂ and its IUPAC name is 30-ethyl-33-(1-hydroxy-2-methylhex-4-enyl)-1,4,7,10,12,15,19,25,28-nonamethyl-6,9,18,24-tetrakis(2-methylpropyl)-3,21-di(propan-2-yl)-1,4,7,10,13,16,19,22,25,28,31-undecazacyclotritriacontane-2,5,8,11,14,17,20,23,26,29,32-undecone (CAS No. 59865-13-3). Its molecular weight is 1203 Daltons. It has the following chemical structure:

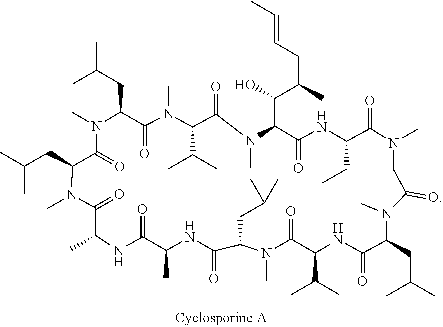 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Attachment of CsA via the hydroxyl group to a spacer provides a CsA-based prodrug where the intact structure of CsA is provided as a drug to the ocular surface after cleavage from the spacer. Since the free hydroxyl group has a low reactivity due to steric hindrance, an amine or thiol group may be provided in place of the hydroxyl group (ignoring any inversion of sterochemistry at the carbon atom alpha to the hydroxyl group upon replacement with an amine or thiol group).

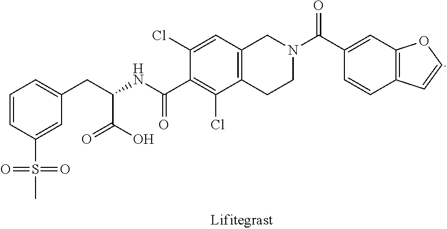 [SMILES: CS(=O)(=O)c1cccc(C[C@H](NC(=O)c2c(Cl)cc3c(c2Cl)CCN(C(=O)c2ccc4ccoc4c2)C3)C(=O)O)c1]

In some embodiments, a drug moiety is lifitegrast (N-{[2-(1-Benzofuran-6-ylcarbonyl)-5,7-dichloro-1,2,3,4-tetrahydro-6-isoquinolinyl]carbonyl}-3-(methylsulfonyl)- L-phenylalanine).

In some embodiments, a drug moiety is atropine ((RS)-(8-Methyl-8-azabicyclo[3.2.1]oct-3-yl) 3-hydroxy-2-phenylpropanoate)).

In some embodiments, a drug moiety is an antibiotic such as azithromycin, clarithromycin, amoxicillin, ampicillin, bacitracin, trovafloxacin, mafenide, sulfacetamide, sulfamethizole, sulfasalazine, sulfisoxazole, trimethoprim, cotrimoxazole, dapsone (diaminodiphenyl sulfone (DDS), thiazolsulfone, acetosulfone.

In some embodiments, a drug moiety is a transient receptor potential cation channel subfamily M (melastatin) member 8 (TRPM8) agonist. In some embodiments, a TRPM8 agonist is selected from linalool (3,7-dimethylocta-1,6-dien-3-ol), geraniol ((2E)-3,7-dimethylocta-2,6-dien-1-ol), hydroxy-citronellal, icilin (3-(2-hydroxyphenyl)-6-(3-nitrophenyl)-3,4-dihydropyrimidin-2(1H)-one), Frescolat MGA 49-methyl-6-propan-2-yl-1,4-dioxaspiro[4.5]decan-2-yl)methanol), Frescolat ML ([(1R,2S,5R)-5-methyl-2-propan-2-ylcyclohexyl] 2-hydroxypropanoate), PMD 38 (2-(2-hydroxypropan-2-yl)-5-methylcyclohexan-1-ol), Coolact P (isopulegol), and Cooling Agent 10 (menthoxypropanediol).

In some embodiments, a drug moiety is a steroidal compound, such as 21-acetoxypregnenolone, alclomethasone, algestone, amcinonide, beclomethasone, budesonide, chloroprednisone, clobetasol, clobetasone, clocortolone, corticosterone, cortisone, cortivazol, deflazacort, desonide, desoximethasone, diflorasone diflucortolone, difluprednate, ethynylestradiol, estradiol, fluazacort, flunisolide, fluocinolone acetonide, fluocinonide, fluocortolone, fluorometholone, fluticasone propionate, fluperolone acetate, fluprednidene acetate, flurandrenolide, formocortal, halcinonide, halobetasol propionate, halopredone acetate, hexacetonide, hydrocortamate, loteprednol, mazipredone, medrysone, meprednisone, mestranol, momethasone, furoate, moxestrol, prednicarbate, prednisolamate, prednisone, prednival, rimexolone, or triamcinolone.

In some embodiments, a drug moiety is an anti-inflammatory agent, such as amfenac, α-bisabolol, bromfenac, bromosaligenin, diflunisal, ditazol, etofenamate, fendosal, fepradinol, glycol salicylate, ibuprofen, ibuproxam, isoxicam, lornoxicam, meloxicam, mesalamine, oxaceprol, oxyphenbutazone, parsalmide, perisoxal, olsalazine, piroxicam, salacetamide, tenoxicam, tiaramide, or tinoridine.

In some embodiments, a drug moiety is an analgesic, such as acetaminophen, acetaminosalol, aminochlorthenoxazin, anileridine, benzylmorphine, bucetin, buprenorphine, butorphanol, capsaicine, ciramadol, codeine, desomorphine, dezocine, dihydrocodeine, dihydromorphine, dimepheptanol, eptazocine, ethoxazene, ethylmorphine, eugenol, floctafenine, glafenine, hydromorphone, hydroxypethidine, p-lactophenetide, levorphanol, meptazinol, metazocine, metopon, morphine, norlevorphanol, normorphine, oxycodone, oxymorphone, pentazocine, phenazocine, phenocoll, phenoperidine, phenylsalicylate, phenylramidol, salicylamide, tiorphan, or tramadol.

### Compositions

Compositions of the present disclosure can include (e.g., comprise, consist essentially of, or consist of) an effective amount, such as a pharmaceutically effective amount, of a prodrug of the present disclosure, including isomers, salts, or solvates thereof, and a carrier, such as a pharmaceutically effective carrier, for administration to an ocular region of a subject to treat an ocular surface condition (e.g., disease).

A composition of the present can have a pH outside a physiological pH (e.g., a physiological pH of 7.4 to 7.9). For example, a composition can have a pH of less than 7.4 in order to protonate a nucleophilic nitrogen atom of a prodrug of the present disclosure effectively hindering its nucleophilic capability until it is under a higher, physiological pH during use in a subject's eye. In some embodiments, a composition of the present disclosure has a pH of about 4 to about 7.4, such as about 5 to about 7, such as about 5 to about 6.

The compositions may be in the form of a liquid (e.g., an ophthalmic drop), a suspension, a gel, a slurry, an ointment, a cream, an emulsion, a solid, a powder of variable sizes macro to nano particle sized (wettable powder or dry powder), or a pellet. In particular aspects, the composition is a liquid composition.

Compositions include one or more pharmaceutically acceptable carriers. A pharmaceutically acceptable carrier may be any suitable carrier. In some embodiments, the carrier may be any one or more of water, an aqueous solution (e.g., saline), a polymer such as hydroxypropyl methylcellulose (hypromellose or HPMC), petrolatum, mineral oil, carboxymethyl cellulose, polyvinyl alcohol, hydroxypropyl cellulose, hyaluronic acid (hyaluronan or HA), glycerin, polyethylene glycol (PEG) such as Polyethylene Glycol 400 (PEG 400), propylene glycol (PG), polysorbate 80, povidone, and/or dextran.

A composition can have a prodrug content (by weight or by volume) of about 0.001% to about 0.05%, such as about 0.001% to about 0.01%, alternatively about 0.01% to about 0.05%, alternatively about 0.05% to about 10%, such as about 0.05% to about 1%, or about 0.05% to about 0.5%, or about 0.3% to about 0.8% or about 0.4% to about 1.2%, or about 0.6% to about 1.5%, or about 1% to about 2%, or about 3% to about 4%. As mentioned above, a prodrug can be present in an amount such that an amount of the drug compound (after release from the prodrug) is present in a reduced amount, as compared a composition comprising the drug compound itself. This advantage provides reduction or elimination of adverse effects provided by high concentrations of a drug compound, such as irritation of the ocular surface. Accordingly, in some embodiments, a composition can have a prodrug content (by weight or by volume) of about 0.001% to about 0.05%, such as about 0.001% to about 0.01%, alternatively about 0.01% to about 0.05%, alternatively about 0.005% to about 0.05%, such as about 0.01% to about 0.045%, such as about 0.02% to about 0.04%, such as about 0.03% to about 0.04%, alternatively about 0.02% to about 0.03%. In some embodiments, a composition can have a drug molecule content (e.g., drug moiety D of a prodrug)(by weight or by volume) of about 0.001% to about 0.05%, such as about 0.001% to about 0.01%, alternatively about 0.01% to about 0.05%, alternatively about 0.005% to about 0.05%, such as about 0.01% to about 0.045%, such as about 0.02% to about 0.04%, such as about 0.03% to about 0.04%, alternatively about 0.02% to about 0.03%.

### Pharmaceutically Acceptable Carriers

The carriers (e.g., pharmaceutically acceptable carriers) of the present disclosure will allow the one or more prodrugs and drug compounds thereof to remain efficacious (e.g., capable of treating an ocular surface disease, such as, dry eye). Examples of carriers can include liquids, suspensions, gels, ointments, nanosized drug particles, pellets, slurries, or solids (including wettable powders or dry powders). The selection of the carrier material can depend on the intended treatment.

Examples of carriers can include such vehicles as water; saline, organic solvents, alcohols, glycols, glycerin, aliphatic alcohols, mixtures of water and organic solvents, mixtures of water and alcohols (e.g., ethanol or isopropanol), mixtures of organic solvents such as alcohol and glycerin, lipid-based materials such as fatty acids, acylglycerols, oils, mineral oils, fats of natural or synthetic origin, phosphoglycerides, sphingolipids, waxes, DMSO, protein-based materials such as collagen and gelatin, volatile and/or non-volatile silicon-based materials, cyclomethicone, dimethiconol, dimethicone copolyol (Dow Corning, Midland, Mich., USA), hydrocarbon-based materials such as petrolatum and squalane, sustained-release vehicles such as microsponges and polymer matrices, suspending agents, emulsifying agents, and other vehicles and vehicle components that are suitable for administration to the ocular region, as well as mixtures of topical vehicle components as identified above or otherwise known to the art.

An advantage of prodrugs of the present disclosure is that the prodrugs can be applied topically as a simple saline solution. In some embodiments, the carrier is a saline solution, for example, phosphate buffer saline, a sodium chloride solution, Ringer's solution (sodium chloride, potassium chloride, calcium chloride and sodium bicarbonate), lactated Ringer's solution (sodium lactate added), BSS Sterile Irrigating Solution® (comprising potassium, calcium, lactate, magnesium and acetate citrate), or BSS+ Sterile Irrigating Solution® (bicarbonate, dextrose, and glutathione added).

In some embodiments, the carrier is a polymer. Examples of acceptable polymers can include, hydroxyethyl cellulose, hydroxypropyl methyl cellulose, carboxymethyl cellulose (e.g., cellulose, or Gum Cellulose), polyethylene oxide, or dextrans. In at least one embodiment, the carrier is hydroxypropyl methyl cellulose (HPMC) (also referred to as hypromellose).

A composition can have a carrier material content (by weight or by volume) of about 99% to about 99.999%, such as about 99.95% to about 99.995%, or about 99.95% to about 99.99%, or about 99.97% to about 99.99% or about 99.97% to about 99.98%, based on total weight of carrier material whether one or more types of carrier material, unless otherwise specified.

### Additional Compounds

The compositions provided herein may include one or more additional compounds, or be used contemporaneously (e.g., used separately but with the compositions of the present disclosure) with, one or more additional compounds. Additional compounds may include antibiotics, steroids, anti-inflammation agents, analgesics, surfactants, chelating agents, buffering agents, pH adjusting agents, adjuvants, or combinations thereof. The additional compounds can provide any purpose, so long as the additional compounds are suitable for use in a composition used on a subject. Beneficial purposes of additional compounds may include synergistic effects (e.g., a greater than additive effect) when combined with the active ingredients (drug compound and/or prodrug) of the composition, enhanced delivery of the drug compound or prodrug to the subject, ease of formulating, or combinations thereof.

In some embodiments, the additional compound is atropine or an atropine derivative.

In some embodiments, an additional compound can be present in the composition in an amount of about 0.0005 wt % to about 10 wt %, such as about 0.05 wt % to about 5 wt %, or about 0.1 wt % to about 3 wt %, or about 0.5 wt % to about 0.8 wt %, or about 0.7 wt % to about 4 wt % based on the total weight of the composition.

### Antibiotics

In some embodiments, the compositions may include at least one antibiotic. The antibiotic may be any antibiotic suitable for use in a subject, in particular a mammalian subject, and more particularly, in a human subject. Examples of antibiotics that may be used with the compositions of the present disclosure include amikacin, gentamicin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, clozacillin, dicloxacillin, flucozacillin, meziocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, oflazacin, trovafloxacin, mafenide, sulfacetamide, sulfamethizole, sulfasalazine, sulfisoxazole, trimethoprim, cotrimoxazole, demeclocycline, soxycycline, minocycline, oxytetracycline, tetracycline, vancomycin, and salts thereof. Additionally, the antibiotics may include any sulfone such as dapsone (diaminodiphenyl sulfone (DDS)) or any dapsone derivative, such as amino acid amides of dapsone, PROMIN™ (sodium glucosulfone), DIASONE™ (sulfoxone sodium), SULPHETRONE™ (solapsone), PROMIZOLE™ (thiazolsulfone), or PROMACETIN™ (acetosulfone). In some embodiments, a sulfone such as dapsone may be administered to an ocular region of a subject with at least one prodrug, or isomer, salt, or solvate thereof. The sulfone and at least one prodrug may be administered in the same composition or in separate compositions, and may be administered simultaneously or sequentially one to the other.

### Steroidal Compounds

In some embodiments, the compositions may further include at least one steroid. The steroid may be any steroid suitable for use in a subject, in particular a mammalian subject, and more particularly, in a human subject. Examples of steroids that may be used with the compositions of the present disclosure include 21-acetoxypregnenol one, acetonide, alclomethasone, algestone, amcinonide, beclomethasone, betamethasone, budesonide, chenodeoxycholic acid, chloroprednisone, clobetasol, clobetasone, clocortolone, cloprednol, corticosterone, cortisone, cortivazol, deflazacort, desonide, desoximethasone, dexamethasone, diflorasone diflucortolone, difluprednate, ethynylestradiol, estradiol, fluazacort, flucloronide, flumethasone, flunisolide, fluocinolone acetonide, fluocinonide, fluocortyn butyl, fluocortolone, fluorometholone, fluticasone propionate, fluperolone acetate, fluprednidene acetate, fluprednisolone, flurandrenolide, formocortal, halcinonide, halobetasol propionate, halomethasone, halopredone acetate, hexacetonide, hydrocortamate, hydrocortisone, loteprednol etabonate, mazipredone, medrysone, meprednisone, mestranol, methylprednisolone, mitatrienediol, momethasone furoate, moxestrol, paramethasone, prednicarbate, prednisolone, prednisolone 25-diethylaminoacetate, prednisolone sodium phosphate, prednisone, prednival, prednylidene, rimexolone, triamcinolone, triamcinolone, tixocortol, triamcinolone, ursodesoxycholic acid, and salts thereof.

### Anti-Inflammatory Agents

In some embodiments, the compositions may further include at least one anti-inflammatory agent. The anti-inflammatory agent may be any anti-inflammatory agent suitable for use in a subject, in particular a mammalian subject, and more particularly, in a human subject. Examples of anti-inflammatory agents can include aceclofenac, acemetacin, acetylsalicylic acid, 5-amino-acetylsalicylic acid, alclofenac, alminoprofen, amfenac, bendazac, bermoprofen, α-bisabolol, bromfenac, bromosaligenin, bucloxic acid, butibufen, carprofen, cinmetacin, clidanac, clopirac, diclofenac sodium, diflunisal, ditazol, enfenamic acid, etodolac, etofenamate, felbinac, fenbufen, fenclozic acid, fendosal, fenoprofen, fentiazac, fepradinol, flufenamic acid, flunixin, flunoxaprofen, flurbiprofen, glucametacin, glycol salicylate, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, naproxen, niflumic acid, oxaceprol, oxaprozin, oxyphenbutazone, parsalmide, perisoxal, phenyl acetylsalicylate, olsalazine, pyrazolac, piroxicam, pirprofen, pranoprofen, protizinic acid, salacetamide, salicilamide O-acetic acid, salicylsulphuric acid, salsalate, sulindac, suprofen, suxibuzone, tenoxicam, tiaprofenic acid, tiaramide, tinoridine, tolfenamic acid, tolmetin, tropesin, xenbucin, ximoprofen, zaltoprofen, zomepirac, tomoxiprol or sulindac, and salts thereof.

### Analgesics

In yet other aspects, the compositions may further include at least one analgesic. The analgesic may be any analgesic suitable for use in a subject, in particular a mammalian subject, and more particularly, in a human subject. Examples of analgesics can include acetaminophen (i.e., paracetamol), acetaminosalol, aminochlorthenoxazin, acetylsalicylic 2-amino-4-picoline acid, acetylsalicylsalicylic acid, anileridine, benoxaprofen, benzylmorphine, 5-bromosalicylic acetate acid, bucetin, buprenorphine, butorphanol, capsaicine, cinchophen, ciramadol, clometacin, clonixin, codeine, desomorphine, dezocine, dihydrocodeine, dihydromorphine, dimepheptanol, dipyrocetyl, eptazocine, ethoxazene, ethylmorphine, eugenol, floctafenine, fosfosal, glafenine, hydrocodone, hydromorphone, hydroxypethidine, ibufenac, p-lactophenetide, levorphanol, meptazinol, metazocine, metopon, morphine, nalbuphine, nicomorphine, norlevorphanol, normorphine, oxycodone, oxymorphone, pentazocine, phenazocine, phenocoll, phenoperidine, phenylbutazone, phenyl salicylate, phenylramidol, salicin, salicylamide, tiorphan, tramadol, diacerein, actarit, or salts thereof.

### Surfactants/Wetting Agents

In some aspects, the compositions may also include at least one surfactant or wetting agent. The surfactant may be a cationic surfactant, amphoteric surfactant, zwitterionic surfactant, or nonionic surfactant. If the surfactant is nonionic, it may be selected from polysorbates, poloxamers, alcohol ethoxylates, ethylene glycol-propylene glycol block copolymers, fatty acid amides, alkylphenol ethoxylates, or phospholipids.

### Chelating Agents

In some embodiments, the compositions may also include a chelating agent, such as edetate salts, like edetate disodium, edetate calcium disodium, edetate sodium, edetate trisodium, or edetate dipotassium.

### Buffering Agents

In some embodiments, the compositions may also include at least one buffer. Examples of buffers may include phosphates (e.g., sodium phosphate, sodium dihydrogen phosphate, disodium hydrogen phosphate, potassium phosphate, potassium dihydrogen phosphate, or dipotassium hydrogen phosphate), borates (e.g., sodium borate, potassium borate) citrates (e.g., sodium citrate, disodium citrate), acetates (e.g., sodium acetate, potassium acetate), or carbonates (e.g., sodium carbonate, sodium hydrogen carbonate).

### pH Adjusting Agents:

In some embodiments, the compositions may also include at least one pH adjusting agent. Examples of pH adjusting agents include sodium hydroxide, potassium hydroxide, sodium carbonate, hydrochloric acid, phosphoric acid, citric acid, or acetic acid.

### Preservatives

In some embodiments, the compositions may be preservative free or may also include at least one preservative. Examples of preservatives include p-hydroxy benzoate esters, benzalkonium chloride, benzethonium chloride, chlorobutanol, benzyl alcohol, sorbic acid or its salts, chlorhexidine gluconate, sodium dehydroacetate, cetylpyridinium chloride, or alkyldiaminoethylglycine hydrochloride. Other compounds that may be included in the compositions can include oleic acid, 1-methyl-2 pyrrolidone, 2,2-dimethyl octanoic acid and N,N dimethyl lauramide/propylene glycol monolaureate or combinations thereof, which may be included for example to minimize the barrier characteristics of the upper most layer of the corneal and conjunctival surfaces, thus, improving efficacy.

### Adjuvants

In some embodiments, the compositions may also include one or more adjuvants. Examples of adjuvants include phosphatidic acid, sterols such as cholesterol, aliphatic amines such as stearylamine, saturated or unsaturated fatty acids such as stearic acid, palmitic acid, myristic acid, linoleic acid, oleic acid, and salts thereof.

### Methods of Administering Prodrugs and Compositions

Further disclosed are methods for treating at least one ocular surface disease comprising administering to an ocular region of a subject one or more of the prodrugs or compositions thereof of the present disclosure.

In some embodiments, methods for treating at least one ocular surface disease includes administering a composition having a pharmaceutically effective amount of at least one prodrug to the ocular region of a subject.

In some embodiments, the composition is a composition that delivers at least one prodrug (including isomers, salts, and solvates thereof) (with a corresponding desired therapeutically effective amount of a drug compound) to the ocular region of a subject to be treated. It is envisioned that the therapeutically effective amount of the prodrug could be greater than 1 wt. % depending what can be tolerated by the subject being treated and the clinical effect(s) at the site of action (ocular surface anatomical structures, including the cornea, conjunctiva, lid margin epithelium, blood vessels, the meibomian gland/sebaceous gland complex, etc.). As provided throughout, the prodrug used to carry out the methods of the present disclosure can be any suitable prodrug or isomer, salt, or solvate thereof. In particular embodiments, the prodrug has a drug moiety that is a cyclosporine A or an isomer, salt or solvate thereof.

In some embodiments, the methods of the present disclosure treat front of the eye ocular surface diseases. In some embodiments, the methods of the present disclosure treat back of the eye ocular surface diseases. In still other embodiments, the methods of the present disclosure treat both front of the eye diseases and back of the eye diseases.

Some examples of front of the eye ocular surface diseases include inflammation, diffuse lamellar keratitis, corneal diseases, edemas, or opacifications with an exudative or inflammatory component, diseases of the eye that are related to systemic autoimmune diseases, any ocular surface disorders from dry eye (including ADDE, EDE, and chronic dry eye generally, keratoconjunctivitis, such as vernal keratoconjunctivitis, atopic keratoconjunctivitis, and sicca keratoconjunctivitis), lid margin diseases, meibomian gland disease or dysfunction, dysfunctional tear syndromes, anterior and or posterior blepharitis, microbial infection, computer vision syndrome, conjunctivitis (e.g., persistent allergic, giant papillary, seasonal intermittent allergic, perennial allergic, toxic, conjunctivitis caused by infection by bacteria, fungi, parasites, viruses or Chlamydia), conjunctival edema anterior uveitis and any inflammatory components or components of the aqueous fluid, inflammatory conditions resulting from surgeries such as LASIK®, LASEK®, refractive surgery, intraocular lens implantation (IOL), irreversible corneal edema as a complication of cataract surgery, edema as a result of insult or trauma (physical, chemical, pharmacological, etc), genetic diseases of the cornea (corneal dystrophies including keratoconus, posterior polymorphous dystrophy; Fuch's dystrophies (corneal and endothelial), etc.), aphakic and pseudophakic bullous keratopathy, scleral diseases with or without inflammatory components, ocular cicatrcial pemphigoid, or pterygium.

Some examples of back of the eye diseases include diseases of the optic nerve (including its cellular and sub cellular components such as the axons and their innervations), glaucomas (including primary open angle glaucoma, acute and chronic closed angle glaucoma and any other secondary glaucomas), myopic retinopathies, macular edema (including clinical macular edema or angiographic cystoid macular edema arising from various etiologies such as diabetes, exudative macular degeneration and macular edema arising from laser treatment of the retina), diabetic retinopathy, age-related macular degeneration, retinopathy of prematurity, retinal ischemia and choroidal neovascularization and like diseases of the retina, genetic disease of the retina and macular degeneration, pars planitis, Posner Schlossman syndrome, Bechet's disease, Vogt-Koyanagi-Harada syndrome, hypersensitivity reactions, toxoplasmosis chorioretinitis, inflammatory pseudotumor of the orbit, chemosis, conjunctival venous congestion, periorbital cellulitis, acute dacryocystitis, non-specific vasculitis, sarcoidosis, or cytomegalovirus infection.

In some embodiments, a method of the present disclosure treats dry eye (including ADDE, EDE, chronic dry eye, etc.).

In some embodiments, compositions of the present disclosure are administered to the ocular region of a subject by topical administration. In at least one embodiment, a method for treating dry eye includes topically administering to an ocular region of a subject a composition comprising a prodrug of the present disclosure, and/or optionally comprising one or more preservatives, and/or optionally comprising one or more compounds for increasing efficacy; and reducing or preventing one or more symptoms or causes of dry eye.

In still other embodiments, the compositions are administered with one or more additional pharmaceutical agents. The one or more additional pharmaceutical agents may be administered, before, after, or simultaneously with the administration of a composition of the present disclosure. In at least one embodiment, the one or more additional pharmaceutical agents is administered before the administration of the compositions of the present disclosure. In another embodiment, the one or more additional pharmaceutical agents is administered after the composition is administered. In still yet another aspect, the one or more additional pharmaceutical agents is administered simultaneously with the administration of a composition of the present disclosure. In embodiments where the one or more additional pharmaceutical agents is administered simultaneously with the administration of the composition, the additional pharmaceutical agent may be formulated with the composition of the present disclosure or administered as a separate pharmaceutical agent at about the same time as the composition of the present disclosure is administered.

Methods can include administering a composition from between 1-8 times daily; and/or from between 1-4 times daily, for 1-4 weeks; and/or from between 1-4 times daily, for up to 4 weeks, then from 1-2 times daily. Alternatively, methods can include administering a composition from between 1-8 times weekly; and/or from between 1-4 times weekly, for 1-4 weeks; and/or from between 1-4 times weekly, for up to 4 weeks, then from 1-2 times weekly. Alternatively, methods can include administering a composition from between 1-8 times monthly; and/or from between 1-4 times monthly, for 1-4 months; and/or from between 1-4 times monthly, for up to 4 months, then from 1-2 times monthly. For some patients, it may be expected that the compositions can be administered indefinitely, permanently, or otherwise on a long-term basis as a maintenance therapy. In some, instances, the method can include administering the compositions of the present disclosure to a subject throughout the lifetime of the subject as a maintenance therapy.

Methods of the present disclosure can be used to prevent and/or reduce one or more symptoms and/or causes of dry eye such as impaired vision, burning sensation, redness, irritation, grittiness, filminess, inflammation, discomfort, pain, chemosis, chalasis, engorged vasculature, anterior lid margin vascularization, zone A posterior lid margin vascularization, or meibomian gland obstruction, secretion, viscosity, turbidity, loss, drop out, or dysfunction. According to methods of the present disclosure, the reducing or preventing of symptoms or causes of dry eye can be quantitatively or qualitatively evidenced by vital staining, such as by lissamine green staining.

In an embodiment, the compositions of the present disclosure are topically administered to the eye to treat dry eye. In a particular embodiment, the compositions are topically administered to the cornea to treat dry eye. In still another embodiment, the compositions are topically administered to the sclera to treat dry eye. In still another embodiment, the compositions are topically administered to the conjunctiva to treat dry eye. In still another embodiment, the compositions are topically administered to the lacrimal sac to treat dry eye. In another embodiment, the compositions are topically administered to the lacrimal canals to treat dry eye. In still another embodiment, the compositions are topically administered to the lacrimal ducts to treat dry eye. In yet another embodiment, the compositions are topically administered to the canthus to treat dry eye. In still another embodiment, the compositions are topically administered to the eyelids to treat dry eye.

In one embodiment, the compositions of the present disclosure are topically administered by administering a liquid (e.g., ophthalmic drops as a composition of the present disclosure) to the ocular region of a subject for example to treat dry eye. In yet another embodiment, the compositions are topically administered by administering a suspension to the ocular region of a subject for example to treat dry eye. In another embodiment, the compositions are topically administered by administering a cream to the ocular region of a subject for example to treat dry eye. In still another embodiment, the compositions are topically administered by administering an emulsion to the ocular region of a subject for example to treat dry eye. In yet another embodiment, the compositions are topically administered by administering a gel to the ocular region of a subject for example to treat dry eye. In still yet another embodiment, the compositions are topically administered by administering a paste, pellet, ointment, spray, or nanoparticle vehicle to the ocular region of a subject for example to treat dry eye. In some embodiments, the composition comprises xanthan gum. In yet still another embodiment, the compositions are topically administered by administering a gel to the ocular region of a subject for example to treat dry eye. In another particular embodiment, the compositions are topically administered by administering an ointment to the ocular region of a subject for example to treat dry eye. In still another embodiment, the compositions are topically administered by administering a particle (e.g., a nanosized or macrosized particle, pellet, etc.) to the ocular region of a subject for example to treat dry eye. In yet another particular aspect, the compositions are topically administered by administering a slurry to the ocular region of a subject for example to treat dry eye.

The administering process can be performed by any method known in the art (e.g., liquid dropper, nanoparticle vehicles, gum materials (e.g., xanthan gum materials), sprays, application of the compositions to a material worn over the eye, such as a patch, contact lenses, etc.). Further, the process of administering the compositions may be repeated as necessary (e.g., more than once, as in the administering process is repeated twice, three times, four times, five times, six times, seven times, eight times, nine times, ten times, eleven times, twelve times, thirteen times, fourteen times, fifteen times, sixteen times, seventeen times, eighteen times, nineteen times, twenty times, etc.) until the ocular surface disease is considered treated.

In some embodiments, the prodrug/composition, alone or in combination with other active agents, can be injected subconjunctivally as well as subtarsally into the eye lids and/or Meibomian glands directly.

The compositions of the present disclosure can be packaged in various package forms for use in the field of topical ophthalmics. In at least one embodiment, the composition is packaged in sterile, preservative-free single-use packs or vials or containers (e.g., the unit dose vials). Each vial, for example as small as a 0.9 mL, may be made of low density polyethylene so as to contain a small quantity of the composition, e.g., 0.2-0.4 for a single use. This way, where the pharmaceutical composition is sterilized and contained in disposable single-dose containers for topical use in drop form, multiple vials in the form of a set of 30 vials, 60 vials and so on can be packaged in a tray with a lid, for example, a polypropylene tray with an aluminum peelable lid. The entire contents of each tray can be dispensed intact, and one vial or pack is used each time and immediately discarded after each use. For example, plastic ampules or vials or containers can be manufactured using blow-fill-seal (BFS) technology. The BFS processes may involve plastic extrusion, molding, aseptic filling, and hermetic sealing in one sequential operation and those processes are known in the art. In another embodiment, the formulation is packaged in multi-dose vials such that the materials can be dispensed as sterile at each time using specialized container/closure maintaining the sterility integrity. In yet another embodiment, the formulation is packed in conventional vials/containers as sterile product.

In some embodiments, the dosage form of the composition is eye drops of heterogeneous aqueous solution, eye drop formulations containing two or more active ingredients in which the first active ingredient is a prodrug of the present disclosure and a second active ingredient is selected from the group consisting of a lymphocyte function-associated antigen antagonist, a corticosteroid, a sodium channel blocker, a non-steroidal anti-inflammatory drug, an antibiotic, and a combination of two or more thereof. For example, an eye drop composition can contain brimonidine or brimonidine tartrate and loteprednol, or brimonidine or brimonidine tartrate and lifitegrast, or brimonidine or brimonidine tartrate and sodium channel blocker, or brimonidine or brimonidine tartrate and an NSAID, or brimonidine or brimonidine tartrate and an antibiotic. In some embodiments, the dosage form of the composition is eye drops of heterogeneous aqueous solution, eye drop formulations containing two or more active ingredients in which the first active ingredient is a prodrug and a second active ingredient is selected from a lymphocyte function-associated antigen antagonist, an alpha 2 adrenergic agonist, a sodium channel blocker, an NSAID, an antibiotic or a combination of two or more thereof. For example, an eye drop composition can contain loteprednol and brimonidine or brimonidine tartrate, or loteprednol and lifitegrast, or loteprednol and a sodium channel blocker, or loteprednol and an NSAID, or loteprednol and an antibiotic. In some embodiments, eye drops can contain aqueous/oily suspensions of the active ingredients in pharmaceutically acceptable carriers and/or excipients. In some embodiments, the mean particle size of the active ingredient employed is about 20 μm or less, such as 10 μm or less, such as 1 μm or less, such as 0.5 μm or less, such as 0.2 μm or less, such as 0.15 μm or less.

Any of the above-described compositions can be used for treating an eye disorder specified herein by administering to a subject (e.g., a human patient) in need of such a treatment a therapeutically effective amount of a given composition such as nanoemulsion or aqueous solution. Some eye disorders for treatment include a dry eye syndrome (e.g., sjogren's syndrome, meibomian gland dysfunction and keratoconjunctivitis); ocular graft-versus-host-di se ase; ocular rosacea; allergic conjunctivitis; autoimmune ocular surface disease; thygeson's superficial punctuate keratopathy; herpes zoster keratitis; Stevens-Johnson syndrome; keratitis; conjunctivitis; blepharitis; blepharochalasis; conjunctivochalasis; blepharoconjunctivitis; blepharokeratoconjunctivitis; post-operative inflammation or pain from ocular surgery; scleritis; episcleritis; anterior uveitis; iritis; cyclitis; ocular surface vascular disorder; ulcerative keratitis; photokeratitis; dacryocystitis; eyelid disorder; congenital alacrima; xerophthalmia; dacryoadenitis; vernal keratoconjunctivitis; pinguecula; and ocular surface disorder induced by chemical burns, thermal burns, or physical insult to the ocular surface eyelid inflammation, pain and/or edema. In some embodiments, the composition is administered topically to an eye of said subject. The composition is formulated as a homogeneous ophthalmic aqueous formulation, a heterogeneous ophthalmic aqueous solution, a hydrogel, or an ophthalmic cream.

### Contacts

In some embodiments a prodrug of the present disclosure is functionalized to a surface of a contact (instead of being functionalized during use to a protein of an ocular surface). For example, a prodrug of the present disclosure having a functional moiety (e.g., where T of Formula (I), (II), or (III) is a hydroxyl group, a thiol group, a boronic acid group, or an amine group) can be coupled with a contact having a functional group such as surface hydroxyl groups or acyl groups. In some embodiments, a contact comprises polyvinyl alcohol content, polyvinyl pyrrolidone content, and/or pHEMA content, at a surface of the contact and is treated with a prodrug of the present disclosure to form a contact coupled with the prodrug.

### EXAMPLES

Platform: A pH dependent drug delivery system as illustrated in Scheme 1 that represents a new chemical entity and has 3-4 individual components: 1) a drug 2) a pH dependent releasable linker 3) a water solubilizing moiety, and 4) a thiol group to enable, muco-adhesion on the ocular surface. The platform can be applied to a variety of drug conjugates, particularly drug conjugates that can be tethered by an ester linkage (such as Cyclosporine A, Azithromycin, TRPM8 agonist, etc.). Mechanistic details of the pH dependent drug delivery system utilizing Cyclosporine A (CsA) are highlighted in Scheme 1. Protonation of the basic amine under a mildly acidic environment, renders the pH dependent drug delivery system unreactive and stable; however, upon exposure to a physiological environment, such as the ocular surface (pH 7.4), a free amine is generated and available for intramolecular nucleophilic addition to the CsA-O-ester carbonyl. Under this action, CsA (or drug) will be chemically liberated and available for pharmacological target engagement in the eye.

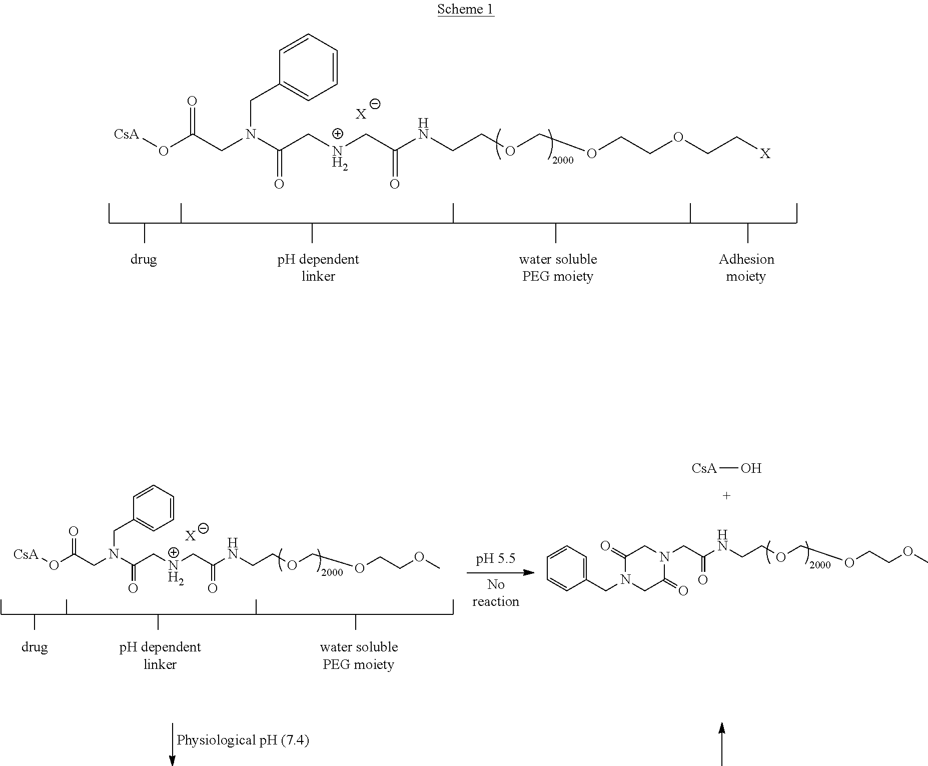

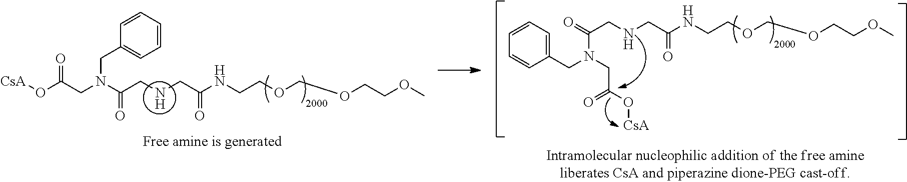

The half-life of a prodrug for release of the drug moiety can be tuned. PEG-CsA I analog shown above may have a t_{1/2}=˜90 min. This half-life may be potentially too long for an intended use. To address this, a tunable pH dependent linker system was envisioned that uses different amine pKa values (7.07-3.85) to produce a “tunable” range of drug release rate profiles for evaluation, as shown in Scheme 2.

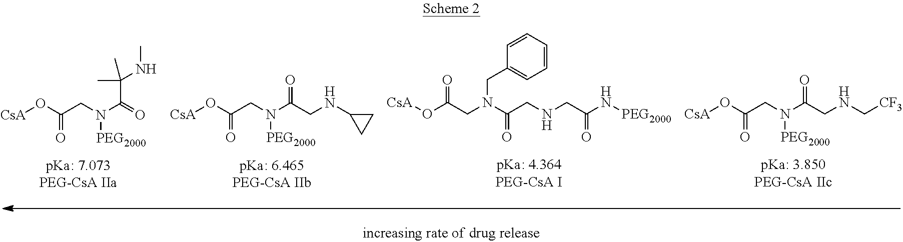

### EXAMPLES

| | |
|---|---|
| 4-DMAP: | 4-dimethylaminopyridine |
| cat.: | catalytic |
| CsA: | cyclosporine A |
| DCM: | dichloromethane |
| DMSO: | dimethyl sulfoxide |
| EDC-HCl: | 1-etyhl-3-(3-dimethylaminopropyl)carbodiimide hydrochloride |
| LC-MS: | liquid chromatography coupled with mass spectrometry |
| NEt3: | triethylamine |
| NMR: | nuclear magnetic resonance spectroscopy |
| PEG: | poly ethylene glycol |
| PBS | Phosphate buffered saline |
| ppm: | parts per million |
| r.t.: | room temperature |
| RI: | refractive index |
| SEC: | size exclusion chromatography |
| THF: | tetrahydrofuran |

A derivative of cyclosporine A containing a pH sensitive linker group following to protocols found in (Khavari et al. 2000). The carboxylic group containing precursor “Cyclosporine-A-(N-benzyl)glycine-linker-OH” was subsequently used for reaction with a 2000 Da PEG-amine. Deprotection with trifluoroacetic acid (TFA) leads to the targeted pH-sensitive-CsA-PEG-2K derivative (I). The structures of all precursors and the synthesized CsA derivative (I) were verified by NMR ¹H, LC-MS, HPLC including determination of yields and purity.

In the second part of the project the synthesized derivative (I) was further characterized. The water solubility was determined by photo analysis of the dissolved derivative and subsequent photometric measurement of supernatants. It was observed that PEGylation has a positive influence on the water solubility by increasing solubility to 120 mg/ml (measurable concentration, maximal concentration may be higher). The water solubility was similar to the non pH-sensitive CsA-PEG-2K derivative which was synthesized and characterized in a previous project.

To test the pH-sensitivity the half-life time of the derivative was analyzed by incubation in PBS pH 7.4 at 37° C. In this experiment a half-life time of approximately 39 h was determined.

In addition a one month stability study at pH 5.5 (estimated storage conditions) was performed at two temperatures (25° C., 40° C.). After 28 days storage at 25° C.; 40° C. approximately 47.3%; 75.5% of the substance was degraded into free CsA and the PEG containing byproduct. A lyophilized sample stored at 25° C. showed no degradation after 21 days so that the degradation process seems to be driven by aqueous hydrolysis. Therefore the stability test revealed that the substance is not stable in solution at pH 5.5.

### Materials & Methods

All chemicals were purchased from commercial sources and were used, unless otherwise stated, without further purification. Solvents were dried as described in the literature. All reactions were carried out under an atmosphere of nitrogen unless otherwise noted.

¹HNMR and ¹³C NMR spectra were recorded on a Bruker ARX 400 (400 MHz/100 MHz) spectrometer at room temperature. Chemical shifts were reported in parts per million (ppm) and the solvent residual peak was used as internal reference: ¹H-NMR (CHCl₃: 7.26 ppm), ¹³C-NMR (CDCl₃: 77.16 ppm). For analysis of CsA-PEG-conjugates a water suppression pulse program was used after optimization to suppress the dominant peak of the polymer backbone in ¹H-NMR spectra resulting in a better signal-to-noise ratio for signals belonging to the CsA core.

Mass spectra were recorded on Waters micromass zq quadrupole mass spectrometer using electron spray ionization.

### Synthesis of Cyclosporine-A-(N-benzyl)glycinate

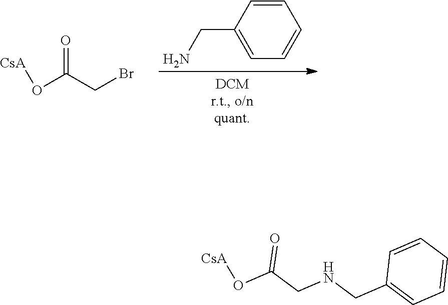

A solution of Cyclosporine-A-O-(2-bromo)acetate* (2.00 g, 1.51 mmol, 1 eq.) in anhydrous dichloromethane was dropwise added under inertgas atmosphere to a stirred solution of benzylamine (0.81 g, 0.83 mL, 7.56 mmol, 5.00 eq.) in anhydrous dichloromethane (20 mL) at room temperature. The reaction was stirred at same temperature overnight upon which a white solid precipitated. After filtration the filtrate was diluted with dichloromethane (50 mL) and was washed with aqueous KHSO₄-solution (3×15 mL, c=5% w/w), saturated aqueous NaHCO₃-solution (2×15 mL) and brine (15 mL). The organic phase was dried over anhydrous sodium sulfate, filtered and the solvent was removed under reduced pressure. The product (2.00 g, 98%) was obtained as a white solid.

* Synthesis of precursor Cyclosporine-A-O-(2-bromo)acetate according to protocols established in a previous study directed to synthesis of a water-soluble CsA-PEG derivative.

Synthesis of Cyclosporine-A-(N-benzyl)glycine-linker-OH

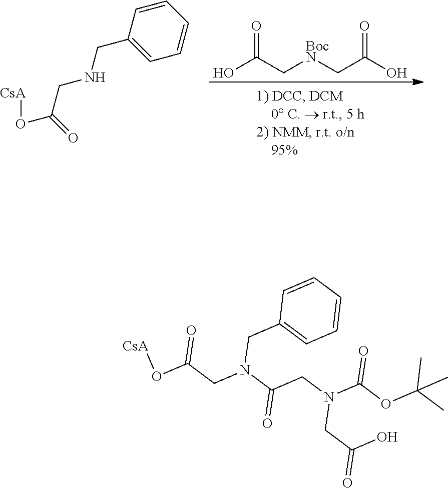

DCC (0.33 g, 1.61 mmol, 1.1 eq.) was added at 0° C. to a stirred solution of N-Boc-iminodiacetic acid (0.376 g, 1.61 mmol, 1.1 eq.) in dichloromethane (10 mL) under argon atmosphere, and the reaction mixture was stirred for 5 h at room temperature. Cyclosporine-A-N-benzylglycinate (1.98 g, 1.47 mmol, 1.0 eq.) and N-Methylmorpholine (0.16 g, 0.18 mL, 1.61 mmol, 1.1 eq.) were added and stirring was continued at room temperature overnight. The precipitate was removed by filtration and the filtrate was diluted with dichloromethane (50 mL). The solution was washed with aqueous KHSO₄-solution (3×15 mL, c=5% w/w), water (15 mL) and brine (15 mL). The organic phase was dried over anhydrous sodium sulfate, filtered and solvent was removed under reduced pressure. The product (2.20 g, 96%) was obtained as a white solid.

Synthesis of Cyclosporine-A-(N-benzyl)glycine-linker-NH-PEG-2K

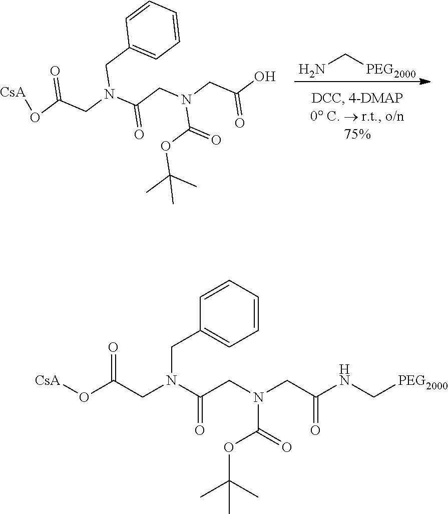

DCC (0.21 g, 1.04 mmol, 1.2 eq.) was added at 0° C. to a stirred solution of Cyclosporine-A-N-benzyl-glycine-linker-OH (1.62 g, 1.04 mmol, 1.2 eq.) in anhydrous dichloromethane (20 mL) under inert gas atmosphere, and stirring was continued for 30 min at same temperature. Amine-PEG-2K (1.73 g, 0.86 mmol, 1.0 eq.) and 4-DMAP (catalytic amounts) were added and the reaction mixture was left stirring overnight at room temperature. The precipitate was removed by filtration and solvent was removed under reduced pressure. The oily was precipitated in n-hexane (50 mL) and the obtained white solid was further purified by MPLC. After freeze drying Cyclosporine-A-N-benzyl-glycine-linker-NH-PEG-2K (2.31 g, 75%) was obtained as a white powder.

Synthesis of Cyclosporine-A-(N-benzyl-glycine)-linker-NH-PEG-2K TFA-salt

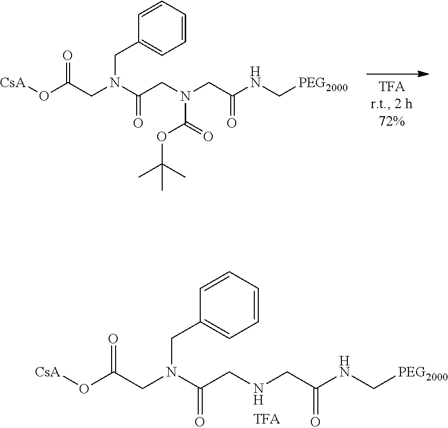

A solution of Cyclosporine-A-N-benzyl-glycine-linker-NH-PEG2000 (0.50 g, 0.14 mmol) in trifluoroacetic acid (1.00 mL) was stirred at room temperature for 2 h.

Solvent was removed under reduced pressure. The oily residue was treated with diethyl ether (50 mL) and stirred for 1 h at room temperature. The precipitate was isolated, washed with diethyl ether (20 mL) and dried overnight at room temperature under reduced pressure. Cyclosporine-A-N-benzyl-glycine-linker-NH-PEG-2K TFA-salt (0.36 g, 72%) was obtained as an off-white solid.

### HPLC Protocols for the Determination of CsA and CsA Derivatives

### HPLC Protocol for Determination of CsA and CsA-Derivative (I) Precursors up to Synthesis Step 3

### Equipment & Reagents:

Merck-Hitachi D7000 HPLC with diode array detector (Merck KGaA, Darmstadt, Germany)
LiChrospher® 100 RP-8 51.tm Column (Merck-Millipore, Darmstadt, Germany)
Eppendorf Thermomixer C (Eppendorf, Hamburg, Germany)
Water & Acetonitrile (gradient, mobile phase, HPLC quality)

### Sample Preparation:

All samples were dissolved in acetonitrile at a concentration of 1 mg/mL
Prior to injection samples were filtered through a 0.45 μm syringe filter

### Applied HPLC Method:

| Solvent gradient | | | |
|---|---|---|---|
| for separation | | | |
| of CsA up to | | | |
| CsA derivative | | | |
| synthesis step 3 | Acetonitrile | Water | Flow |
| Time [min] | [%] | [%] | [ml/min] |
| 0.0 | 30 | 70 | 1 |
| 5.0 | 30 | 70 | 1 |
| 25.0 | 80 | 20 | 1 |
| 35.0 | 80 | 20 | 1 |
| 35.1 | 30 | 70 | 1 |
| 45.0 | 30 | 70 | 1 |

HPLC Protocol for Determination of pH-Sensitive CsA—PEG-2K Derivative (I) Equipment & Reagents:
Merck-Hitachi Chrommaster 5000 HPLC with two channel UV-Vis detector (Merck KGaA, Darmstadt, Germany)
VYdac® Diphenyl 5 μm Column (VYdac, USA)
Eppendorf Thermomixer C (Eppendorf, Hamburg, Germany)
Water & Acetonitrile (gradient, mobile phase, HPLC quality)

### Sample Preparation:

All samples were diluted in acetonitrile at a concentration of 13 mg/mL
Samples were incubated in an Eppendorf Thermomixer C (25° C., 2000 rpm, 45 min)
Prior to injection samples were filtered through a 0.45 μm syringe filter

### Applied HPLC Method:

| Solvent gradient | | | |
|---|---|---|---|
| for separation | | | |
| of pH-sensitive | | | |
| CsA-PEG-2K | Acetonitrile | Water | Flow |
| Time [min] | [%]* | [%]* | [ml/min] |
| 0.0-14.0 | 45 | 55 | 1.5 |
| (isocratic) | | | |
| 14.1-20.0 | 80 | 20 | 1.5 |
| (gradient) | | | |
| 20.1-25.0 | 80 | 20 | 1.5 |
| (isocratic) | | | |
| 25.1-30.0 | 45 | 55 | 1.5 |
| (isocratic) | | | |

Solubility Tests of pH-Sensitive CsA-PEG-2K Derivative (I)

### Sample Preparation

Samples of pH-sensitive CsA-PEG-2K derivative (I) were put into 1.5 ml reaction tubes (Eppendorf, Hamburg, Germany) subsequently dissolved in PBS (pH 5.5) to reach concentrations up to 120 mg/ml. The solubilization of the material was performed in a thermomixer (Thermomixer C, Eppendorf, Hamburg, Germany) at 25° C., 45 min, 2000 rpm. The samples were than centrifuged 10 min at 13.400 rpm to separate potential insoluble compounds.

Photometric Analysis of pH-Sensitive CsA-PEG-2K Derivative (I)

The absorbance maxima for CsA-PEG-2K was determined in a previous study (Synthesis of a water-soluble CsA derivative). Therefore all solutions were measured at 280 nm (absorbance maxima) at 25° C. using PBS (pH 5.5) as control.

Half-life-time of pH-sensitive CsA-PEG-2K derivative (I)

For the determination of half-life time samples were dissolved in PBS pH 7.4 at a concentration of 60 mg/ml. Directly after dissolution a t=0 h sample was taken. Subsequently the samples were incubated at 37° C. in a climatic chamber with constant humidity (55%) for 48 h. Samples were taken at 2 h, 4 h, 6 h, 8 h, 23 h and 48 h.

### Sample Preparation and HPLC Analysis:

50 μl of the stock solution were diluted with 50 μl of a mixture of (water/TFA; 95/5) to stop pH-induced degradation. To this solution 200 μl acetonitrile containing 400 ppm toluene as trace marker was given. Subsequently samples were centrifuged (13.000×g, 15 min, RT) and 100 μl of the supernatant was used for HPLC analysis using the protocol described in 2.5.2. For data analysis the peak intensities of derivative (I) were chosen. The reduction of peak intensities was calculated with the use of a standard curve (7 points) with pH-sensitive PEGylated CsA derivative (I) and expressed relative to the t=0 h value.

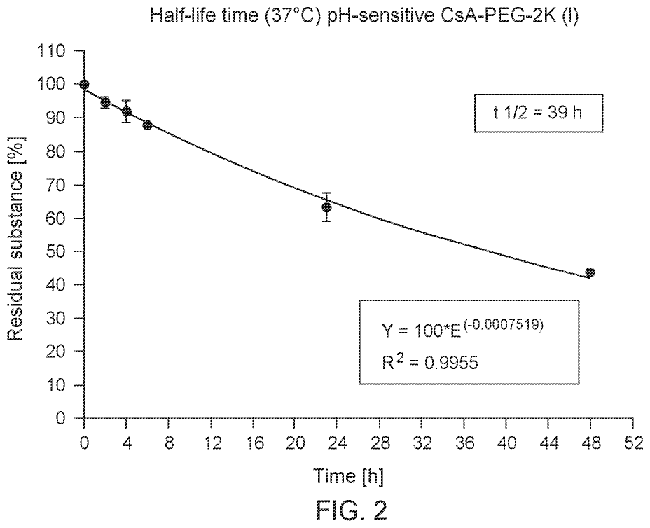

Half-life time was evaluated using GraphPad Prism 6 software (GraphPad, San Diego, Calif.). Results are shown in FIG. 2.

One Month Stability Test of pH-Sensitive-CsA-PEG-2K Derivative (I)

The stability of the substance in aqueous solution was determined by dissolving the material at a concentration of 40 mg/ml in PBS pH 5.5. The solution was subsequently pipetted into 2 ml reaction tubes which were stored at 25° C. and 40° C. in a climatic chamber under constant humidity (55%). For each condition triplicates were prepared. Samples were taken at day 0 (directly after dissolution), day 1, day 3, day 7, day 14, day 21 and day 28 and analyzed by HPLC.

### Sample Preparation and HPLC Analysis:

100 μl of the stock solution were diluted with 200 μl of acetonitrile containing 400 ppm toluene as trace marker. Subsequently samples were centrifuged (13.000×g, 15 min, RT) and 100 μl of the supernatant were used for HPLC analysis using the protocol described above. For data analysis the peak intensities of derivative (I) were chosen. The reduction of peak intensities was calculated by a standard curve (7 points) with pH-sensitive PEGylated CsA derivative (I) and expressed relative to the blank value=day 0. The formed degradation product CsA has a very low solubility and aggregates during the stability study. Therefore only a qualitative analysis of the water soluble fraction could be performed.

### Results and Discussion

Synthesis of pH-Sensitive CsA-PEG-2K Derivative (I)

The targeted compound “pH-sensitive CsA-PEG-2K derivative (I)” was obtained after a 5 step synthesis as TFA salt with approximately 46% end yield and an estimated purity >95% (HPLC).

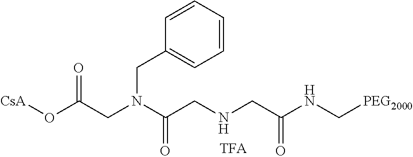

Solubility Tests of PEGylated pH-Sensitive CsA-PEG-2K Derivative (I)

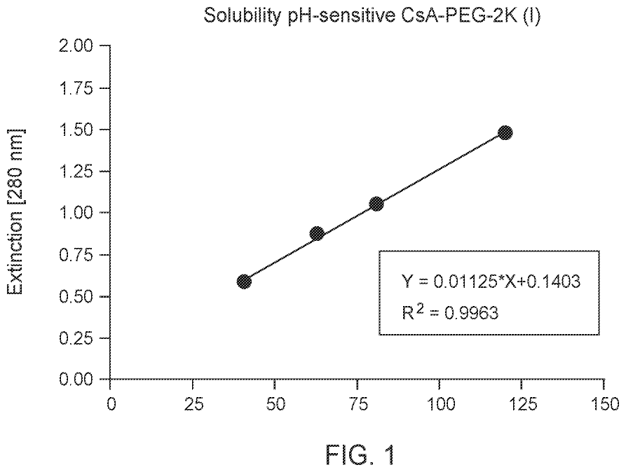

A solubility test was performed in PBS pH 5.5 at 20° C. at 4 different concentrations 40,60,80,120 mg/ml of pH-sensitive CsA-PEG-2K derivative (I). The substance was highly soluble up to 120 mg/ml determined by visual and photometric analysis. Viscosity increased as a function of concentration but the substance still behaved as a liquid. Higher solubility will be possible since no plateau was reached in photometric analysis (FIG. 1). Samples were stored at 4° C. for 2 weeks, centrifuged at maximal speed and then photographed to detect potential particles.

FIG. 1 is a graph illustrating photometrical analysis the solubility of pH-sensitive CsA-PEG-2K derivative (I). Measurement of pH-sensitive CsA-PEG-2K derivative (I) in PBS (pH 5.5) at 280 nm. The measurement results in a concentration curve (Y=0.01125*X+0.1403; R2 =0.9963). Evaluation was performed using GraphPad Prism 6 software (GraphPad, San Diego, Calif.). After two weeks, no visible aggregation of pH-sensitive CsA-PEG-2K derivative (I) was detected for any of the concentrations shown.

Half-Life Time of pH-Sensitive CsA-PEG-2K Derivative (I)

FIG. 2 is a graph illustrating half-life time of pH-sensitive CsA-PEG-2K. Degradation profile of pH-sensitive CsA-PEG-2K derivative (I) at 37° C. The measurement results in a degradation curve which was fitted using a nonlinear regression (one phase decay; Y=100*E(−0.0007519); R2 =0.9955). The calculated half-life time was (t ½=39 h). Evaluation was performed using GraphPad Prism 6 software (GraphPad, San Diego, Calif.). Data represent the mean and standard deviation of three replicates.

One Month Stability Time of pH-Sensitive CsA-PEG-2K Derivative (I)

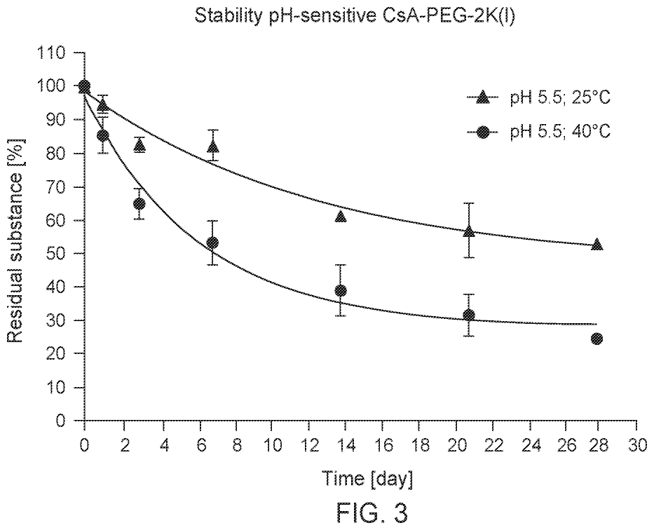

After 28 days storage at 25° C., approximately 47.3% of the substance was degraded into free CsA and the PEG containing byproduct. An increase of the storage temperature to 40° C. results into a faster degradation with 75.5% of the substance degraded after 28 days (see FIG. 3). A lyophilized sample stored at 25° C. showed no degradation after 21 days so that the degradation process seems to be driven by aqueous hydrolysis. Therefore the stability study revealed that the substance is not stable in solution at pH 5.5.

FIG. 3 is a graph illustrating stability of pH-sensitive CsA-PEG-2K derivative (I) at different temperatures. Beside HPLC analysis the substance was also characterized by visual analysis. With progression of the degradation the solution of the substance becomes white and cloudy. The velocity of this process increased with temperature; the solution stored at 25° C. showed no visible change after 7 days whereas the solution stored at 40° C. turns white after 3-7 days. First no aggregates could be detected after centrifugation but with progressing degradation deposits became visible. This behavior may be a hint of potential micellar structures of pH-sensitive CsA-PEG-2K derivative (I) in water. These micelles may first function as an immobilizer for the formed water insoluble CsA but if the concentration of free CsA reaches the maximal capacity deposits are formed.

Overall, prodrugs, compositions, and methods of the present disclosure provide topically administered ophthalmic drug delivery at reduced concentrations, as compared to conventional formulations and improvement of adverse reactions associated with conventional topically administered ophthalmic drug delivery. Prodrugs, compositions, and methods of the present disclosure further provide topically administered ophthalmic drug delivery systems having sustained viability (e.g., release of a drug compound from the prodrug) with reduced frequency of application of the drug compound topically to a patient. In addition, prodrugs of the present disclosure provide reduced “lead in” time even at reduced concentration of drug content.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The foregoing description is provided to enable any person skilled in the art to practice the various embodiments. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. No claim element is to be construed under the provisions of 35 U.S.C. §112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A compound represented by Formula (I): 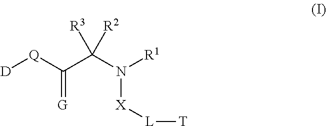
wherein:
D is a drug moiety;
Q is an oxygen atom, a sulfur atom, or a nitrogen atom;X is a chemical bond or 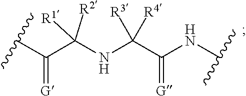
each of G, G′, and G″ is independently an oxygen atom or a sulfur atom;
L is a polyether, polythioether, acetal, ester, hydrazone, imine, orthoester, oxime, phosphoramidate, vinyl ether, or β-thiopropionate;
T is a hydrogen atom, a hydroxyl group, a thiol group, a boronic acid group, or an amine group;
R¹ is selected from the group consisting of a substituted aryl group, an unsubstituted aryl group, and a substituted carbonyl group; and
each of R², R³, R^{2′}, R^{3′}, and R^{4′} is independently a hydrogen atom, a substituted C1-c0 alkyl group, or an unsubstituted C¹-C²⁰ alkyl group.

2. The compound of claim 1, wherein R¹ is selected from the group consisting of:

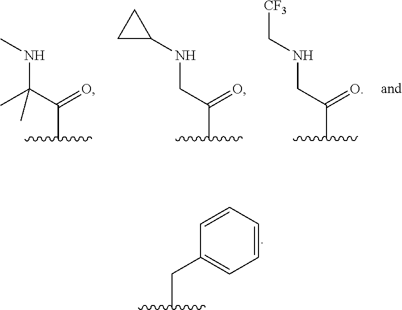

3. The compound of claim 1, wherein the compound is represented by Formula (II): 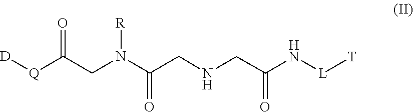
wherein:
D of Formula (II) is a drug moiety;
Q of Formula (II) is an oxygen atom, a sulfur atom, or a nitrogen atom;
L of Formula (II) is a polyether;
T of Formula (II) is a hydrogen atom, a hydroxyl group, a thiol group, a boronic acid group, or an amine group; and
R is a substituted aryl group or an unsubstituted aryl group.

4. The compound of claim 3, wherein R of Formula (II)is an aryl group selected from the group consisting of substituted benzyl, unsubstituted phenyl, substituted benzyl, and unsubstituted benzyl.

5. The compound of claim 3, wherein R of Formula (II) is unsubstituted benzyl.

6. The compound of claim 3, wherein L of Formula (II) is a polyethylene glycol having about 1,500 to about 2,500 ethylene oxide units.

7. The compound of claim 3, wherein T of Formula (II) is a boronic acid group or a thiol group.

8. The compound of claim 3, wherein D of Formula (II) is 30-ethyl-33-(1-hydroxy-2-methylhex-4-enyl)-1,4,7,10,12,15,19,25,28-nonamethyl-6,9,18,24-tetrakis(2- methylpropyl)-3,21-di(propan-2-yl)-1,4,7,10,13,16,19,22,25,28,31-undecazacyclotritriacontane-2,5,8,11,14,17,20,23,26,29,32-undecone or N-{[2-(1-Benzofuran-6-ylcarb onyl)-5,7-dichloro-1,2,3,4-tetrahydro-6-isoquinolinyl]carbonyl}-3-(methylsulfonyl)-L-phenylalanine.

9. The compound of claim 3, wherein D of Formula (II) is (RS)-(8-Methyl-8-azabicyclo[3.2.1]oct-3-yl) 3-hydroxy-2-phenylpropanoate) or azithromycin.

10. The compound of claim 3, wherein D of Formula (II) is selected from the group consisting of:
3,7-dimethylocta-1,6-dien-3-ol,
(2E)-3,7-dimethylocta-2,6-dien-1-ol,
hydroxy-citronellal,
3-(2-hydroxyphenyl)-6-(3-nitrophenyl)-3,4-dihydropyrimidin-2(1H)-one,
9-methyl-6-propan-2-yl-1,4-dioxaspiro[4.5]decan-2-yl)methanol,
[(1R,2S,5R)-5-methyl-2-propan-2-ylcyclohexyl] 2-hydroxypropanoate, and isopulegol.

11. The compound of claim 1, wherein the compound is represented by Formula (III): 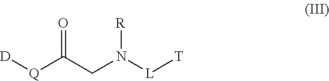
wherein:
D of Formula (III) is a drug moiety;
Q of Formula (III) is an oxygen atom, a sulfur atom, or a nitrogen atom;
L of Formula (III) is a polyether;
T of Formula (III) is a hydrogen atom, a hydroxyl group, a thiol group, a boronic acid group, or an amine group; and
R of Formula (III) is a substituted carbonyl group.

12. The compound of claim 11, wherein R of Formula (III) is a methyl acyl group substituted at the methylene carbon of the methyl acyl group with a secondary amine group.

13. The compound of claim 11, wherein R is represented by formula (IIIa): 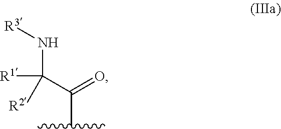
wherein:
each of R^{1′} and R^{2′} is independently selected from the group consisting of a hydrogen atom, a substituted C₁-C₁₀ alkyl group, and an unsubstituted C₁-C₁₀ alkyl group; and
R3′ is selected from the group consisting of a substituted aryl, an unsubstituted aryl, a substituted alkyl, and an unsubstituted alkyl.

14. The compound of claim 13, wherein R^{1′} and R^{2′} are each a hydrogen atom.

15. The compound of claim 14, wherein R^{3′} is a C₁-C₁₀ alkyl group optionally substituted with a halogen atom or a nitro group.

16. The compound of claim 13, wherein R^{1′} and R^{2′} are independently a C₁-C₅ alkyl group.

17. The compound of claim 11, wherein L of Formula (III) is a polyethylene glycol having about 1,500 to about 2,500 ethylene oxide units.

18. The compound of claim 11, wherein T of Formula (III) is a boronic acid group or a thiol group.

19. The compound of claim 11, wherein D of Formula (III) is 30-ethyl-33-(1-hydroxy-2-methylhex-4-enyl)-1,4,7,10,12,15,19,25,28-nonamethyl-6,9,18,24-tetrakis(2- methylpropyl)-3,21-di(propan-2-yl)-1,4,7,10,13,16,19,22,25,28,31-undecazacyclotritriacontane-2,5,8,11,14,17,20,23,26,29,32-undecone or N-{[2-(1-Benzofuran-6-ylcarbonyl)-5,7-dichloro-1,2,3,4-tetrahydro-6-isoquinolinyl]carbonyl}-3-(methylsulfonyl)-L-phenylalanine.

20. The compound of claim 12, wherein D of Formula (III) is (RS)-(8-Methyl-8-azabicyclo[3.2.1]oct-3-yl) 3-hydroxy-2-phenylpropanoate) or azithromycin.

21. The compound of claim 11, wherein D of Formula (III) is selected from the group consisting of:
3,7-dimethylocta-1,6-dien-3-ol,
(2E)-3,7-dimethylocta-2,6-dien-l-ol, hydroxy-citronellal,
3-(2-hydroxyphenyl)-6-(3-nitrophenyl)-3,4-dihydropyrimidin-2(1H)-one,
9-methyl-6-propan-2-yl-1,4-dioxaspiro[4.5]decan-2-yl)methanol,
[(1R,2S,5R)-5-methyl-2-propan-2-ylcyclohexyl] 2-hydroxypropanoate, and isopulegol.
